(19) United States
(12) Patent Application Publication
(10) Pub. No.: US 2026/0167646 A1
(43) Pub. Date: Jun. 18, 2026

(54) BROMODOMAIN INHIBITORS FOR CANCER THERAPY

(71) Applicant: University of Tennessee Research Foundation, Memphis, TN (US)

(72) Inventors: Duane Miller, Collierville, TN (US); Lawrence Pfeffer, Memphis, TN (US); Dong-Jin Hwang, Arlington, TN (US)

(73) Assignee: University of Tennessee Research Foundation, Memphis, TN (US)

(21) Appl. No.: 19/420,072

(22) Filed: Dec. 15, 2025

Related U.S. Application Data
(60) Provisional application No. 63/733,820, filed on Dec. 13, 2024.

(51) Int. Cl. C07D 487/08; A61K 31/167; A61K 31/17; A61K 31/325; A61K 31/40; A61K 31/404; A61K 31/415; A61K 31/495; A61K 31/4995; A61P 35/00; C07C 233/15; C07C 271/58; C07C 275/30; C07C 275/34; C07C 333/08; C07D 207/33; C07D 209/08; C07D 231/12; C07D 519/00

(52) U.S. Cl. CPC C07D 487/08; A61K 31/167; A61K 31/17; A61K 31/325; A61K 31/40; A61K 31/404; A61K 31/415; A61K 31/495; A61K 31/4995; A61P 35/00; C07C 233/15; C07C 271/58; C07C 275/30; C07C 275/34; C07C 333/08; C07D 207/33; C07D 209/08; C07D 231/12; C07D 519/00

(57) ABSTRACT

The present disclosure relates to PFI-3-derived bromodomain inhibitor compounds and their use in treating glioblastoma and other cancers. New small-molecule analogs organized into compound series 2, 3, 4 and 5 selectively target BRG1/BRM bromodomains and sensitize glioblastoma cells to DNA-alkylating agents such as temozolomide. The compounds, their pharmaceutically acceptable salts and prodrugs are provided, together with pharmaceutical compositions comprising at least one such compound optionally in combination with a DNA-alkylating agent. Methods of treating cancer, including temozolomide-resistant glioblastoma, by administering a therapeutically effective amount of at least one of the disclosed compounds alone or in combination with temozolomide are also described.

### CROSS REFERENCE TO RELATED APPLICATION

This application claims the benefit of U.S. Provisional Patent Application Ser. No. 63/733,820, filed Dec. 13, 2024, which is incorporated herein by reference.

### STATEMENT OF FEDERALLY SPONSORED RESEARCH

This invention was made with government support under grant number CA-281977 awarded by National Cancer Institute. The government has certain rights in the invention.

### TECHNICAL FIELD

The presently disclosed subject matter relates to bromodomain inhibitors, including novel selective inhibitors of the Brahma-related gene-1 (BRG1) bromodomain and to methods of using bromodomain inhibitors to treat cancer, particularly therapy-resistant glioblastoma. For example, in some embodiments, the presently disclosed subject matter relates to methods of treating glioblastoma comprising administering a BRI and a therapeutic agent, such as a DNA alkylating agent and/or radiotherapy, to a subject in need of treatment for glioblastoma

### BACKGROUND

Glioblastoma (GBM) is the most common malignant primary brain tumor in adults and a leading cause of cancer-related morbidity and mortality in the United States. Surgical resection of GBM, combined with adjuvant temozolomide (TMZ) chemotherapy and radiation therapy, remains the primary treatment modality, but its 5-year overall prognosis is dismal (<10% survival) which has remained unchanged for decades. The aggressive and diffuse infiltrative nature of GBM makes it extremely difficult to treat. Furthermore, the rapid tumor recurrence after surgical resection and treatment by radiotherapy and chemotherapy demonstrates the ability of the residual GBM cells to evade treatment and propagate. These residual cells are believed to be mainly GBM cancer stem cells (GSCs), which are intrinsically chemoresistant and have many properties of neural stem cells. The use of TMZ alone has limited activity against GBM due to therapeutic resistance that develops over time probably reflective the these intrinsically resistant GSCs.

Bromodomains (BRDs) are a family of evolutionarily conserved motifs identified for the first time in the early 1990s in the brahma gene of Drosophila melanogaster. BRDs are compact protein modules comprising approximately 110 amino acids, which bind to acetylated lysine residues, such as those on the N-terminal tails of histones, and recruit other chromatin factors, thereby regulating gene transcription. There are a total of 61 highly diverse BRDs that have been identified in 46 different BRD-containing proteins encoded by the human genome. Of the 46 known, 11 had two bromodomains, and one protein had 6 bromodomains. They are classified into eight subfamilies according to its sequence homology. BRDs are increasingly being considered as attractive therapeutic targets for a variety of diseases including inflammatory and cardiovascular diseases, and cancer. BRDs play the critical role in control of target genes that are difficult to modulate directly with small molecules.

BRG1 and BRM contain BRDs and are the catalytic subunits of the SNF/SWI chromatin-remodeling complex. BRG1 and BRM play key roles in regulating gene expression and transcription control by disrupting histone-DNA contacts in an ATP-dependent manner. Loss of components of SWI/SNF has been linked to cancer development. BRG1/BRM BRD targeting chemical probes would be useful tools to understand the modulation of SWI/SNF mediated processes, and any inhibitors identified could also offer starting points for druglike lead compounds of potential therapeutic utility in cancers and other diseases. PFI-3 was identified as a broadly selective, potent and cellular active inhibitor of family VIII BRDs including BRG1/BRM/PB1. PFI-3 showed excellent binding affinities for PB1 (Kd 54 nM), and BRG1/BRM (Kd<0.1 μM) and excellent selectivity for these sub-family VIII BRDs in a panel against >40 BRDs from other families. Recently published are the structural-related analogs of PFI-3 that target the BRG1 catalytic subunit of the SWI/SNF complex. Although inhibitory effects were not observed against a range of cellular endpoints in 12 primary human cell-based systems when incubated with PFI-3 and its analogs, it was found that PFI-3 and its analogs enhanced the antiproliferation activity and cell death-inducing effects of temozolomide (TMZ) in both TMZ-sensitive GBM cells and TMZ-resistant GBM cells. In an intracranial GBM animal model, it was also found that PFI-3 and its analogs showed an increase in survival of animals bearing GBM tumors that were treated with TMZ.

Whereas BRG1 and BRM are strongly linked to cancer development and there is a synthetic lethal relationship between BRG1 and BRM, developing novel selective BRG1 and/or BRM BRD inhibitors is needed to explore whether the BRD of BRG1 and/or BRM represents a druggable target in brain tumors. Inhibiting BRG1 by genetic or pharmacologic means promotes glioma cell differentiation, loss of cancer sternness and sensitizes glioma to the effects of alkylating agents, such as TMZ and carmustine, which are standard treatment for GBM patients. Taken together, the BRG1 and/or BRM subunits of SWI/SNF have been identified as novel druggable therapeutical targets in GBM and revealed the therapeutic potential of applying novel small molecule BRG1 inhibitors of SWI/SNF to improve the standard-of-care treatment for GBM.

Accordingly, there is an ongoing need for identifying new therapies and molecular targets in the treatment of GBM, particularly for the development of new therapeutic strategies for targeting therapy-resistant cells in GBM.

### SUMMARY

Presently disclosed are four different series of PFI-3 analogs (compound series 2, 3, 4, and 5), which have been synthesized and examined to determine the activity of each to sensitize GBM cells to TMZ-induced cell death to find the best treatment for GBM. We first optimized the A-ring of series 2, followed by finding the optimum chirality of the bicyclic ring system, which was the R-isomer. This was followed by replacing the bicyclic ring system with a 5-membered linker in series 3, which provided weak activity compared to PFI-3. We also examined the di-phenyl urea compounds of 4, which demonstrated much better action on bromodomain inhibitory activity as compared to PFI-3, especially compound 4a (difluoro phenyl analogs on the A- and B-rings). Finally, methoxyphenyl-B-ring with linker PFI-3 analog 5 showed exceptionally strong activity as a bromodomain inhibitor. Overall, reconfiguration of A- and B-ring substituents in the initial lead compound 2a and optimization of A- and B-rings resulted in a significant boost to bromodomain inhibitor activity for enhancing TMZ in treating GBM. These optimized compounds retain the pharmaceutically druggable properties as potential drug candidates for GBM treatment as calculated by ADME using SwissADME. Notably, this progression was achieved in rational design, synthesis, and proper pharmaceutical tests for possible new drugs like compounds 2a, 2b, 2c, 4a, and 5 for enhancing the action of TMZ in treating GBM.

This Summary is neither intended nor should it be construed as being representative of the full extent and scope of the present disclosure. Moreover, references made herein to “the present disclosure,” or aspects thereof, should be understood to mean certain embodiments of the present disclosure and should not necessarily be construed as limiting all embodiments to a particular description. The present disclosure is set forth in various levels of detail in this Summary as well as in the attached drawings and the Description of Embodiments and no limitation as to the scope of the present disclosure is intended by either the inclusion or non-inclusion of elements, components, etc. in this Summary. Additional aspects of the present disclosure will become more readily apparent from the Description of Embodiments, particularly when taken together with the drawings.

### BRIEF DESCRIPTION OF FIGURES

The subject matter regarded as the invention is particularly pointed out and distinctly claimed in the concluding portion of the specification. The invention may be further understood by reference to the following detailed description when read with the accompanying figures.

FIG. 1 shows the design and optimization of PFI-3 derivatives and representatives of the modified analogues. *: Compound 2as (S, S-type) is the isomer of 2a (R, R) of bridge part.

FIG. 2A and FIG. 2B shows the activity of series 2 compounds to sensitize GBM cells to TMZ-induced cell death compared with PFI-3. FIG. 2A shows series 2 compounds used with TMZ and FIG. 2B shows series 2 compounds without using TMZ. LN229 GBM cells were treated with PFI-3 or the indicated series 2 (2 μM) in combination with TMZ (50 μM). Control cells were treated with vehicle (DMSO). Treatment with TEDs alone had no effect on cell viability. Cell death was determined by ELISA at 72 hr which quantifies histone-complexed DNA fragments. Results were normalized to the cell death-inducing activity of PFI-3 in combination with TMZ.

FIG. 3A and FIG. 3B shows the activity of series 3, 4 and 5 compounds to sensitize GBM cells to TMZ-induced cell death compared with PFI-3. FIG. 3A shows series 3, 4 and 5 compounds used with TMZ and FIG. 3B shows series 3, 4 and 5 compounds without using TMZ. LN229 GBM cells were treated with PFI-3 or the indicated series 3, 4, or 5 (2 μM) in combination with TMZ (50 μM). Control cells were treated with vehicle (DMSO). Treatment with TEDs alone had no effect on cell viability. Cell death was determined by ELISA at 72 hr which quantifies histone-complexed DNA fragments. Results were normalized to the cell death-inducing activity of PFI-3 in combination with TMZ.

FIG. 4A and FIG. 4B summarize the SAR study and considerations for the optimized structures of the series 2 and 4 compounds to sensitize GBM cells to TMZ-induced cell death. FIG. 4A shows the SAR study and considerations for the optimized structures of for the series 2 compounds and FIG. 4B shows the SAR study and considerations for the optimized structures of series 4 compounds. *: Compared with R-type linker with substituents (X_{1,2}═F, X₃═H, and Y₁═OCH₃, Y₂═H), **: Compared with R-type linker with substituents (X_{1,2}═F, X₃═H, and Y_{1,2}═F).

FIG. 5 shows the results of a study of the two compounds (IV-255 and IV-275) show marked anticancer activity by themselves in 3-dimension cell cultures of GBM cancer stem cells (GSCs) in a dose dependent manner.

### DETAILED DESCRIPTION

In the following detailed description, numerous specific details are set forth to provide a thorough understanding of the invention. However, it will be understood by those skilled in the art that the present invention may be practiced without these specific details. In other instances, well-known methods, procedures, and components have not been described in detail so as not to obscure the present invention.

This disclosure provides novel derivatives of the base compound PFI-3 (Formula I):

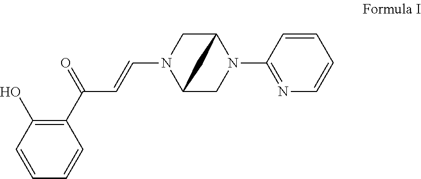 [SMILES: O=C(/C=C/N1C[C@H]2C[C@@H]1CN2c1ccccn1)c1ccccc1O]

In some embodiments, the invention encompasses compounds from the compound series 2 disclosed herein of the Formula II:

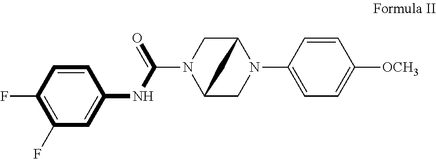

wherein X is NH, NH urea, O, CH₂, S

In some embodiments, the invention encompasses compounds from the compound series 3 disclosed herein of the Formula III:

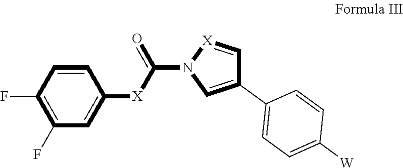

wherein X is CH or N; and W is F or OCH₃.

In some embodiments, the invention encompasses compounds from the compound series 4 disclosed herein of the Formula IV:

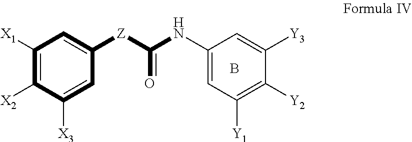

wherein:
X₁, X₂, and X₃ are each independently one of H, F, or OCH₃;
Y₁, Y₂, and Y₃ are each independently one of H or F;
Z is NH, O, S or CH₂; and
the B ring is phenyl or indole

In some embodiments, the invention encompasses compounds from the compound series 5 disclosed herein of the Formula V:

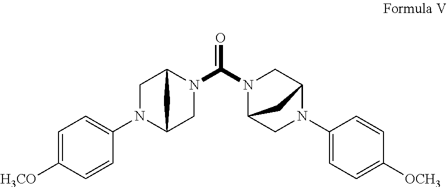

In some embodiments, the invention encompasses compounds from the compound series 5 disclosed herein of the Formula Va:

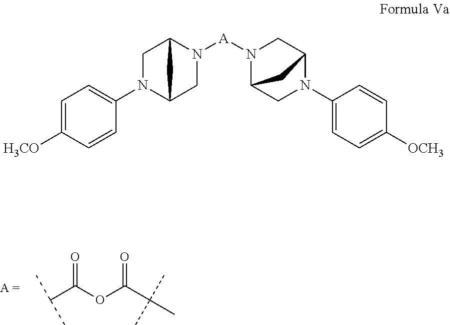

wherein A is as shown.

In some embodiments, the compound used is selected from a group consisting from:

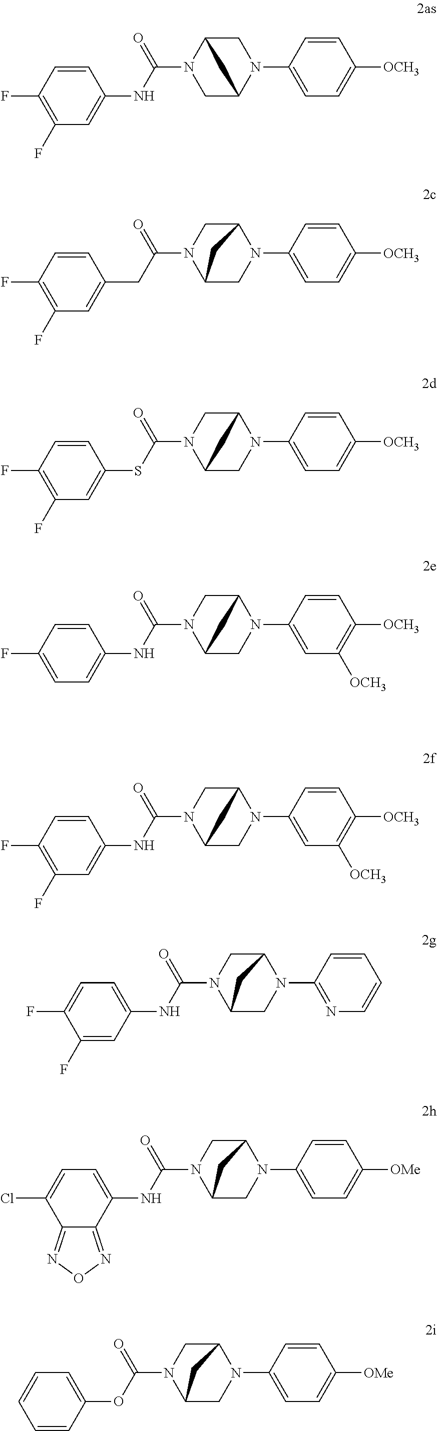

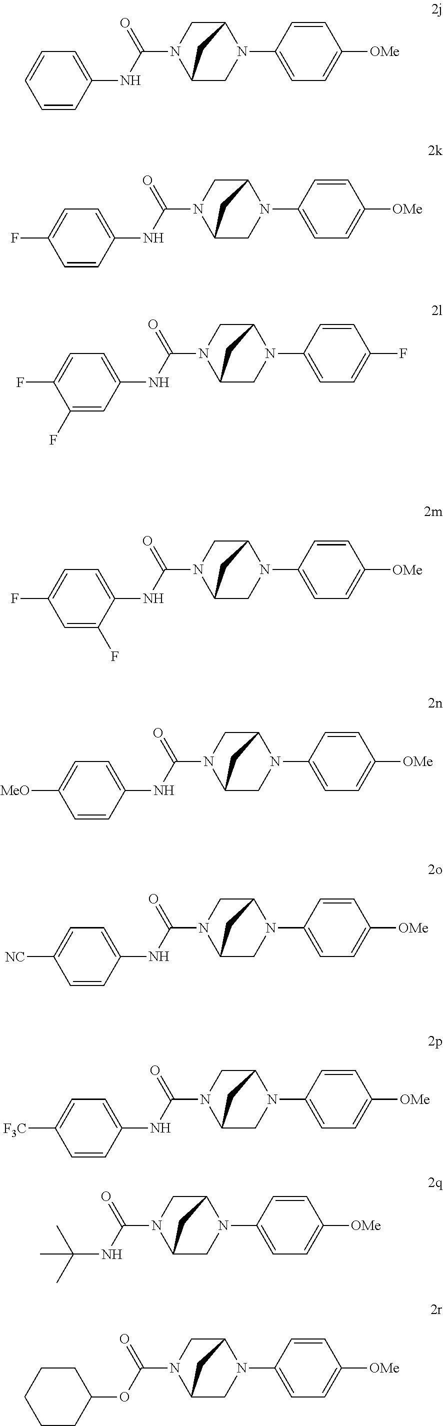

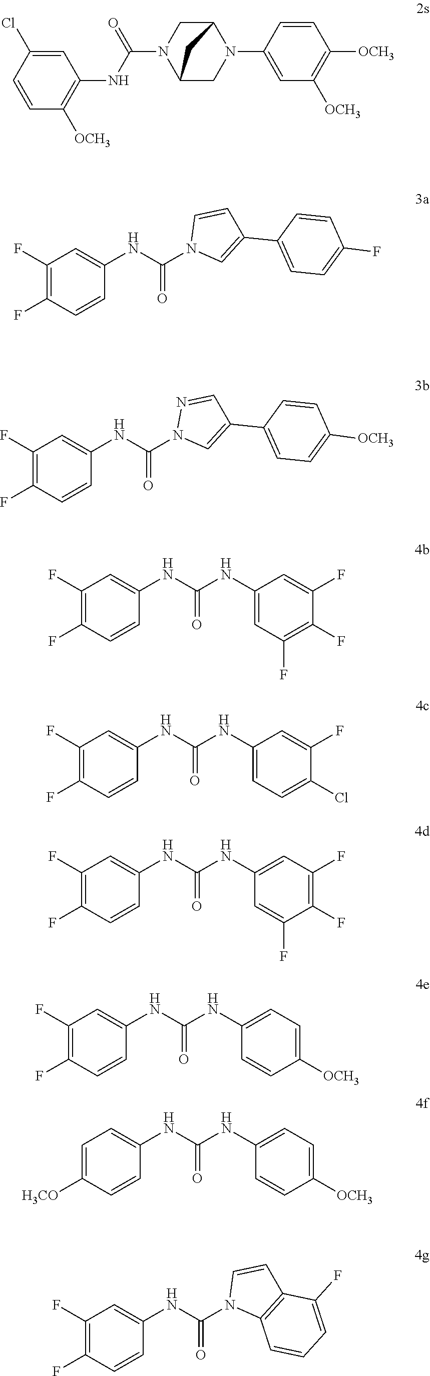

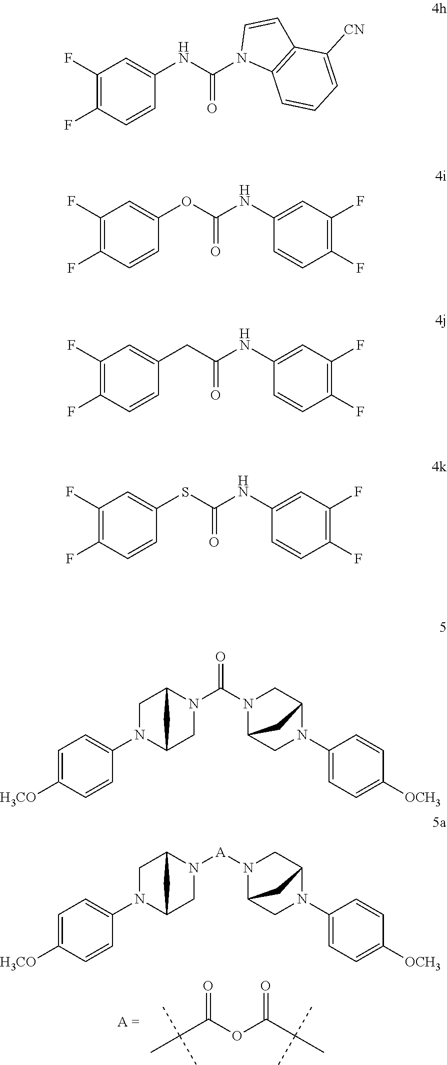

Another aspect of this disclosure provides a pharmaceutical composition comprising at least one compound of this disclosure, or a pharmaceutically acceptable salt thereof, and at least one pharmaceutically acceptable carrier.

Another aspect of this disclosure provides pharmaceutical kits containing a pharmaceutical composition of this disclosure, prescribing information for the composition, and a container.

Another aspect of this disclosure provides methods for inhibiting bromodomain activity in a subject, including administering to the subject a therapeutically effective amount of a bromodomain inhibitor compound of this disclosure, or a pharmaceutically acceptable salt thereof.

Another aspect of this disclosure provides pharmaceutical compositions comprising at least one compound of this disclosure and at least one pharmaceutically acceptable additive.

Another aspect of this disclosure provides pharmaceutical compositions comprising at least one compound of this disclosure and at least one DNA alkylating agent.

A further aspect of this disclosure provides pharmaceutical compositions comprising at least one compound of this disclosure and temozolomide.

Another aspect of this disclosure provides pharmaceutical kits containing a pharmaceutical composition of this disclosure, prescribing information for the composition, and a container.

Another aspect of this disclosure provides methods for inhibiting bromodomain receptors in a subject, including administering to the subject a therapeutically effective amount of a compound of this disclosure, or a pharmaceutically acceptable salt thereof.

This disclosure also provides methods of preventing, treating, or ameliorating cancer, including administering a therapeutically-effective amount of a compound of this disclosure to a subject in need thereof.

This disclosure also provides methods of preventing, treating, or ameliorating glioblastoma, including administering a therapeutically-effective amount of a compound of this disclosure to a subject in need thereof.

In some embodiments, the glioblastoma is a DNA alkylating agent-resistant glioblastoma.

The invention disclosed herein encompasses various embodiments wherein the subject of treatment may be a mammal, including humans. The therapeutic compounds and methods described herein are applicable to both non-human mammals and humans, with the aim of providing effective treatment for a wide range of medical conditions.

For non-human mammalian embodiments, the therapeutic compounds and methods may be employed in veterinary medicine for the treatment of animals such as dogs, cats, horses, cows, pigs, rodents, and other domesticated or wild mammals. These embodiments may involve administering the therapeutic compounds via suitable routes and dosage regimens tailored to the species, size, and health condition of the animal.

For human embodiments, the therapeutic compounds and methods are intended for use in the treatment of various medical conditions in humans where the BRG1 subunit is highly expressed. These conditions may include, but are not limited to, the treatment of cancer.

Compounds described herein are synthesized using any suitable procedures starting from compounds that are available from commercial sources or are prepared using procedures described herein. General methods for the preparation of compounds as described herein are modified by using appropriate reagents and conditions, for the introduction of the various moieties found in the Formulas as provided herein.

In the context of the present invention, the term “patient” or “subject” refers to an individual, whether human or non-human mammal, who is receiving or undergoing treatment with the therapeutic compounds or methods disclosed herein. In human embodiments, the patient may be an individual diagnosed with a medical condition or disease for which treatment is indicated. In non-human mammalian embodiments, the subject may be an animal, such as a dog, cat, horse, rodent, or other mammalian species, for which therapeutic intervention is necessary or desirable. The terms “patient” or “subject” are used interchangeably throughout this specification to refer to the recipient of treatment, regardless of whether they are human or non-human mammals.

“Administration of” or “administering” refers to the act of delivering or applying the therapeutic compound to a patient or subject for the purpose of treating a medical condition or disease. Administration may be performed by healthcare professionals, caregivers, or the patients themselves, whether solely or under the guidance and supervision of qualified personnel.

“Treating” or “treatment” refers to the administration of a therapeutic agent to a patient with the intention of alleviating, curing, or preventing a disease or medical condition. The term may refer to any mode of therapy, including pharmacological, surgical, radiation, or other types of therapy. The term “preventing” may also encompass prophylactic treatment, which is the administration of a therapeutic agent to prevent the onset or recurrence of a disease or medical condition.

A “pharmaceutically acceptable salt” refers to a salt form of a compound that is acceptable for use in pharmaceutical formulations. These salts must be safe for administration to patients and provide the desired pharmacological activity when used in therapeutically effective amounts. Such salts are commonly formed to enhance solubility, stability, bioavailability, or ease of crystallization of the active therapeutic compound without altering its intrinsic therapeutic properties. Pharmaceutically acceptable salts may include those derived from pharmaceutically acceptable inorganic or organic acids and bases. The salt form is typically prepared by reacting the compound with an appropriate acid or base to form a salt. The choice of the acid or base may depend on various factors, including the solubility, stability, and bioavailability of the salt. A pharmaceutically acceptable salt should be non-toxic and should not cause any significant adverse effects in the patient. Examples of pharmaceutically acceptable salts include, but are not limited to, hydrochloride, sulfate, citrate, tartrate, maleate, fumarate, mesylate, besylate, lactate, acetate, benzoate, succinate, and phosphate salts.

Compounds possessing an acidic proton can also be converted into their non-toxic metal or amine addition salt forms by reacting them with suitable organic or inorganic bases. Suitable salt forms include, for example, ammonium salts; alkali and alkaline earth metal salts such as lithium, sodium, potassium, magnesium, and calcium salts; salts formed with organic bases including benzathine, N-methyl-D-glucamine, and hydrabamine; as well as salts with amino acids such as arginine and lysine.

Additionally, certain compounds described herein may also exist in tautomeric forms.

A “prodrug” refers to a pharmacologically inactive compound that is designed to be converted into an active drug in the body. Prodrugs may be advantageous for various reasons, including improving the pharmacokinetic properties of a drug, enhancing its bioavailability, and reducing its toxicity. Prodrugs may be designed to be converted into the active drug through various mechanisms, including hydrolysis, oxidation, reduction, or enzymatic cleavage.

A “pharmaceutical composition” refers to a formulation that comprises one or more active ingredients and one or more pharmaceutically acceptable carriers. The pharmaceutical composition facilitates administration of the compound to a patient or subject. The composition may be in any suitable form, including but not limited to tablets, capsules, powders, solutions, suspensions, emulsions, gels, creams, ointments, patches, and injectable formulations. The choice of a pharmaceutical composition may depend on various factors, including the type and route of administration, the stability of the active ingredient, and the desired therapeutic effect. The composition may be prepared by any suitable method, including but not limited to blending, mixing, granulating, compressing, or lyophilizing.

A “pharmaceutically acceptable carrier” refers to a substance or a combination of substances that are inert, non-toxic, and compatible with the active ingredient(s) of the compound of the present disclosure. The carrier may be a solid, liquid, or a gas and may include, but is not limited to, excipients, diluents, binders, lubricants, disintegrants, fillers, and solvents. The choice of a pharmaceutically acceptable carrier may depend on various factors, including the type and route of administration, the stability of the active ingredient, and the desired therapeutic effect.

A “therapeutically effective amount” refers to a quantity of the active ingredient(s) of the compound of this disclosure that exhibits the desired therapeutic effect in a patient. The amount may vary depending on various factors, including the disease or disorder to be treated, the patient's age and health condition, the route of administration, and the desired therapeutic effect. A therapeutically effective amount may be determined by one skilled in the art using routine experimentation, and may be expressed as a range or a specific value.

“DNA alkylating agents” refer to a class of chemotherapeutic compounds designed to disrupt the normal functioning of DNA within cells. These agents achieve their therapeutic effect by introducing intra-strand or inter-strand DNA cross-links that prevent proper separation of DNA strands during replication, resulting in structural and functional damage that inhibits critical processes such as replication and transcription. DNA alkylating agents are particularly effective against rapidly dividing cells, making them suitable for the treatment of aggressive cancers, including glioblastoma. However, it is known that cancers can become resistant to DNA alkylating agents. Temozolomide is an example of a DNA alkylating agent used to treat glioblastoma.

The pharmaceutical composition of the present invention can be administered through various routes to achieve therapeutic effects tailored achieve the desired therapeutic effect. These routes include oral, parenteral, rectal, topical, nasal, buccal, vaginal, and inhalation administration. Orally administered pharmaceutical compositions are ingested through the mouth and are typically in the form of tablets, capsules, solutions, or suspensions, offering convenient and widely accepted dosing options. Dosage forms and strengths can vary depending on factors such as patient age, severity of inflammation, and desired therapeutic outcome. Parenteral administration involves delivering the pharmaceutical composition directly into the body through means other than the digestive tract, such as subcutaneous (SC), intravenous (IV), intramuscular (IM), intraperitoneal (IP), or intrathecal routes. This route bypasses the gastrointestinal system, allowing for rapid absorption and systemic distribution of the therapeutic agent making it suitable for acute conditions or situations requiring immediate therapeutic intervention. Pharmaceutical compositions for intravenous administration are typically in the form of sterile solutions or suspensions. Rectal administration involves the insertion of suppositories, enemas or rectal gels into the rectum for localized or systemic effects. This route of administration is particularly useful for patients unable to take oral medications or requiring local treatment of conditions such as hemorrhoids or inflammatory bowel disease. Topical administration entails applying creams, ointments, gels, or patches directly onto the skin or mucous membranes for targeted localized treatment while minimizing systemic side effects. Nasal administration involves delivering sprays or drops into the nasal cavity for systemic or local effects. Buccal administration involves placing tablets or patches between the cheek and gum for absorption through the buccal mucosa. Vaginal administration involves the insertion of suppositories, creams, or tablets into the vagina for local or systemic effects. Inhalation administration involves inhaling aerosols, powders, or vapors into the respiratory tract for rapid absorption and distribution of the therapeutic agent. Each route of administration offers unique advantages in terms of efficacy, convenience, and patient compliance, allowing for tailored treatment approaches to optimize therapeutic outcomes.

Dosage and administration regimens may vary depending on factors such as the patient's age, weight, medical condition, and response to treatment. The dosage range may be from about 10 mg to about 1000 mg per day, depending on the disease or disorder to be treated, the patient's age and health condition, and the desired therapeutic effect. The appropriate dosage and administration schedule should be determined by a qualified healthcare professional based on individual patient characteristics and therapeutic goals.

The following abbreviations were used in this specification:
ADME Absorption, Distribution, Metabolism and Excretion
Calcd Calculated
ESI Electrospray ionization
EtOAc Ethyl acetate
GBM Glioblastoma
LCMS Liquid chromatography/mass spectrometry
m/z Mass-to-charge ratios
NOE Nuclear Overhauser effect
TED Therapeutic-enhancing drugs
tₘᵢₙ Time in minutes
TLC Thin-layer chromatography
TMZ Temozolomide
t_{R} Time resolution

### EXAMPLES

The following Examples are presented to more fully illustrate the preferred embodiments of the invention. They should in no way, however, be construed as limiting the broad scope of the invention. The following methods were used to conduct the experiments described in Examples 1-47, below:

### Biological Reagents and Cell Cultures

U87, U251 and LN229 (ATCC, Manassas, VA) GBM cell lines were grown in DMEM containing 10% fetal bovine serum (Hyclone, Logan, UT) supplemented with penicillin (100 IU/ml) and streptomycin (100 μg/ml) at 37° C. with 5% CO₂. The cells were authenticated by short-tandem repeat analysis.

For stem cell growth assays, GBM cells were plated in nonadherent 96 well plates (1×104 cells/well) and grown in supplemented neural basal media. At one day of plating cells were treated with two of the lead compounds, and the number of tumor spheres (colonies) were enumerated after 7 days of culture.

### Cell Death Assays

For cell death assays, cells were plated into 48 well plates (1×104 cells/well), and after 3 days of drug treatment the levels of apoptosis in the attached cells were determined according to the instructions using the cell death ELISAPLUS assay (Roche), which measures cytoplasmic histone-associated DNA fragments.

### General Chemistry Methods

All chemicals for synthesis were purchased from Sigma-Aldrich Chemical Co., Fisher Scientific (Pittsburgh, PA, USA), Ambeed, Inc. (Arlington Height, IL), Combi-Blocks, Inc. (San Diego, CA), 1Pluschem Product List (San Diego, CA) etc. and used without further purification. Moisture-sensitive reactions were carried under an argon atmosphere. Analytical thin-layer chromatography (TLC) was performed on pre-coated silica gel (Merck Kieselgel 60 F254 layer thickness 0.25 mm). A Bruker Avance III 400 (Billerica, MA) spectrometer obtained NMR spectra. Chemical shifts are observed as parts per million (ppm) relative to TMS in CDCl₃ or DMSO-d₆. The structure of synthesized compounds was also utilized by 1H-1H 2D-COSY and 2D-NOE NMR analytic methods. The use of silicagel (230-400 mesh, Merck) for Flash column chromatography was utilized. A Bruker Esquire-LC/MS system (Bruker Daltonics, Billerica, MA) equipped with electrospray/ion trap instrument in positive and negative ion modes (ESI source). The purity of the final compounds was examined by an Agilent 1100 HPLC system (Santa Clara, CA). HPLC conditions: 45% acetonitrile at flow rate of 1.0 mL/min using a LUNA 5μ C18 100A column (250×4.60 mm) purchased from Phenomenex (Torrance, CA) at ambient temperature. The UV detection was set at 340 nm or 245 nm. The properties of the compounds were established by careful integration of areas for all peaks detected and determined as more than 95% for all compounds tested for biological study.

### Example 1

### Design and Synthesis of New Urea, Carbamate Methylene-Amide and Carbamothioate Type PFI-3 Derivatives Compound Series 2, 3, 4 and 5

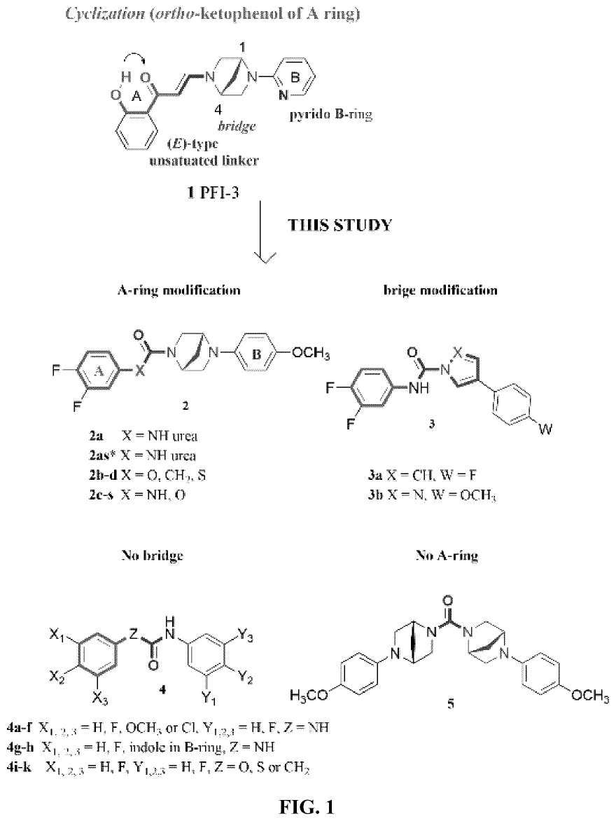

Based on previous findings with the first generation of TEDs, additional SAR was performed which led to a generation of new analogs as shown in FIG. 1. Structural modifications were made in the A-ring (ortho-keto-phenol form) and B-ring, and in the linker and bridged portion, which included cyclized part A (the ortho-keto-phenol analogs 2, 3 and 4 in FIG. 1) and B-rings, linker, and bridged fragments.

As shown in FIG. 1, analogs of the PFI-3 bromodomain inhibitor were made by modification of substituted phenol A-ring part and pyrido-B-ring, which resulted in improved GBM cell death-inducing activity of TMZ in compounds 9f and 11d as compared to PFI-3. Based on the findings gained from the structural biology study, re-scaffolding studies were conducted resulting in the discovery of a new generation of bromodomain inhibitors. Disclosed herein are newly developed TEDs, whose modifications are described below:
i. cyclization of ortho-keto-phenol of the A-ring part of PFI-3 to convert to substituted benzene ring.
ii. connected with a set of new linkers, which is the urea (for compound 2a), carbamate (for compound 2b) methylene-amide (for compound 2c), and carbamothioate (for compound 2d) instead of unsaturated linker in PFI-3.
iii. (1R, 4R)-type isomer (for compound 2a) bridge tested with (1S, 4S) isomer (for compound 2as).
iv. multi (or mono)-substituted A or B-ring shown in FIG. 1 (for compounds 2e-s).
v. as skeleton diversification, a 5-membered ring on the bridge part of PFI-3 was focused on for compounds 3a and 3b and no bridge (for compounds 4a-k) and no A-ring derivative (for compound 5) of PFI-3.

### Example 2

### Synthesis of Series 2 Compounds

The synthesis of target compounds 2a-b and 2 h-q were prepared through the general synthetic route below shown in Scheme 1. Reagents and conditions: (a) BINAP, Pd(OAc)₂, t-BuONa, toluene, reflux; (b) i. EtOH, AcCl, 0° C.-rt, ii. Et₃N, DCM, rt; (c) bis(trichloromethyl) carbonate (BTC, Triphosgene), (3,4-difluoroaniline (for compound 2a), 3,4-difluorophenol (for compound 2b) or 3,4-difluorobenzenethiol (for compound 2s)), Et₃N, DCM, 0° C.-rt; (d) DCM, Et₃N, 0° C.-rt.

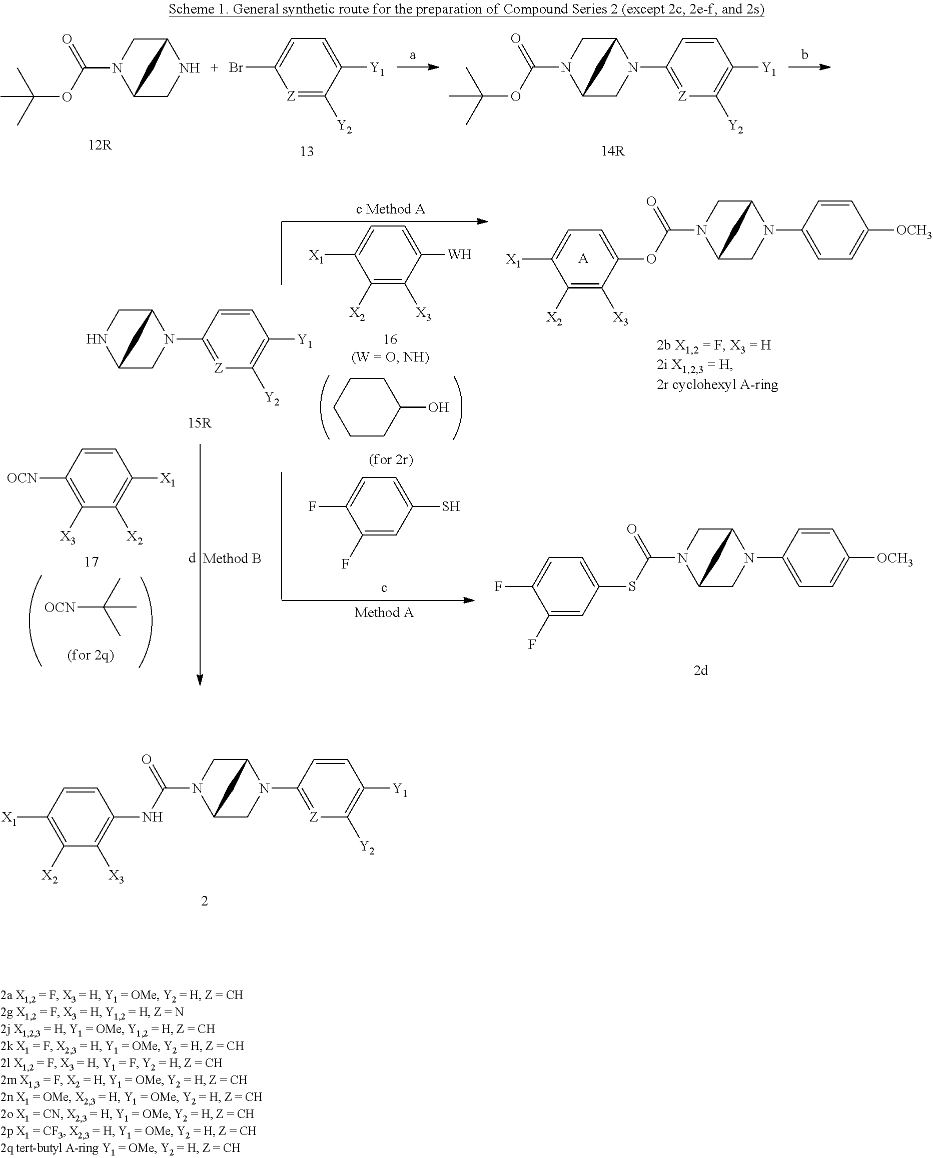

Urea (i.e. compounds 2a, 2e-h etc.), carbamate (i.e. compounds 2b, 2i, 2r) and carbamothioate compound 2d were synthesized as shown in Scheme 1. Two synthetic methods A and B, as detailed in Example 6, from free amine 15R with corresponding aromatic isocyanate 17 or aromatic aniline 16 via bis(trichloromethyl) carbonate (BTC) mediated with 3,4-difluoroaniline were utilized to prepare urea compound 2a as shown in Scheme 1. Compound 2as (isomer of compound 2a) was prepared with the initialization of S-isomer of 12R through the same method with the compound 2a procedure. The combination of substituted phenol 16 and 15R was employed to produce to carbamates (compounds 2b, 2i and 2r). Carbamothioate compound 2d produced from 15R and commercial 3,4-difluorobenzenethiol via BTC activation as the same way.

The synthesis of target compounds 2c, 2e-f, and 2s were prepared through the general synthetic route below shown in Scheme 2. Reagents and conditions: (a) i. EtOH, AcCl, 0° C.-rt, ii. Et₃N, DCM, rt; (b) SOCl₂, THF, 0° C. to rt; (c) Et₃N, DCM, rt, 0° C.-rt; (d) bis(trichloromethyl) carbonate (BTC or Triphosgene), Et₃N (or pyridine), DCM, rt; (e) DCM, Et₃N (or pyridine), rt.

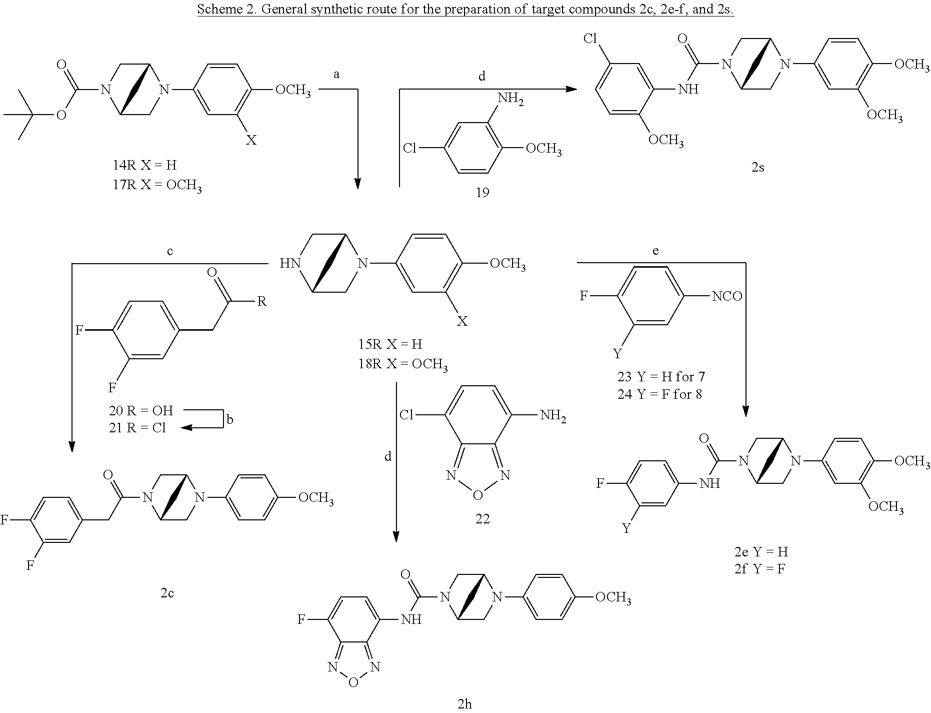

The essential intermediate 15R was synthesized from protected chiral 14R by the acid-condition deprotected synthetic method. The protected 14R was prepared by the Buchwald-Hartwig reaction using Pd(OAc)₂ and BINAP to catalyze the cross-coupling of protected amine 12R with aryl bromide 13. The preparation of aliphatic A-ring compounds (2q and 2r) was conducted by using 2-isocyanato-2-methylpropane (for compound 2q) and cyclohexanol (for compound 2r) instead of 17 or 16 as reactant each through the same procedure as shown in Scheme 1.

Methylene amide compound 2c and several urea (i.e. compounds 2e-f, 2 h, 2s) were summarized in Scheme 2. Protected amines 14R or 17R prepared by the Buchwald-Hartwig reaction, was used as starting materials to produce designed compounds (2c, 2e-f, 2s) as shown in Scheme 2. The protected amine 14R or 17R produced to free amine 15R or 18R by deprotection mediated acidic solution. The reaction of compound 15R with activated acid chloride 21 generated target methylene amide compound 2c under basic conditions. For compounds 2 h and 2s, bis(trichloromethyl) carbonate mediated reaction of free amine 14R and corresponding anilines 19 and 22 produced the target compounds 2 h and 2s under basic conditions using triethylamine or pyridine at room temperature. The synthesis of compounds 2e and 2f was achieved through isocyanate 23 and 24 with corresponding aniline 15R and 18R at room temperature.

### Example 3

### Synthesis of Compounds 3 and 4 Series

The synthesis of target compounds 3a-b and 4a-4k were prepared through the general synthetic route below shown in Scheme 3. Reagents and conditions: (a) DCM, Et₃N, rt.; (b) bis(trichloromethyl) carbonate (BTC or Triphosgene), Et₃N, DCM, rt.

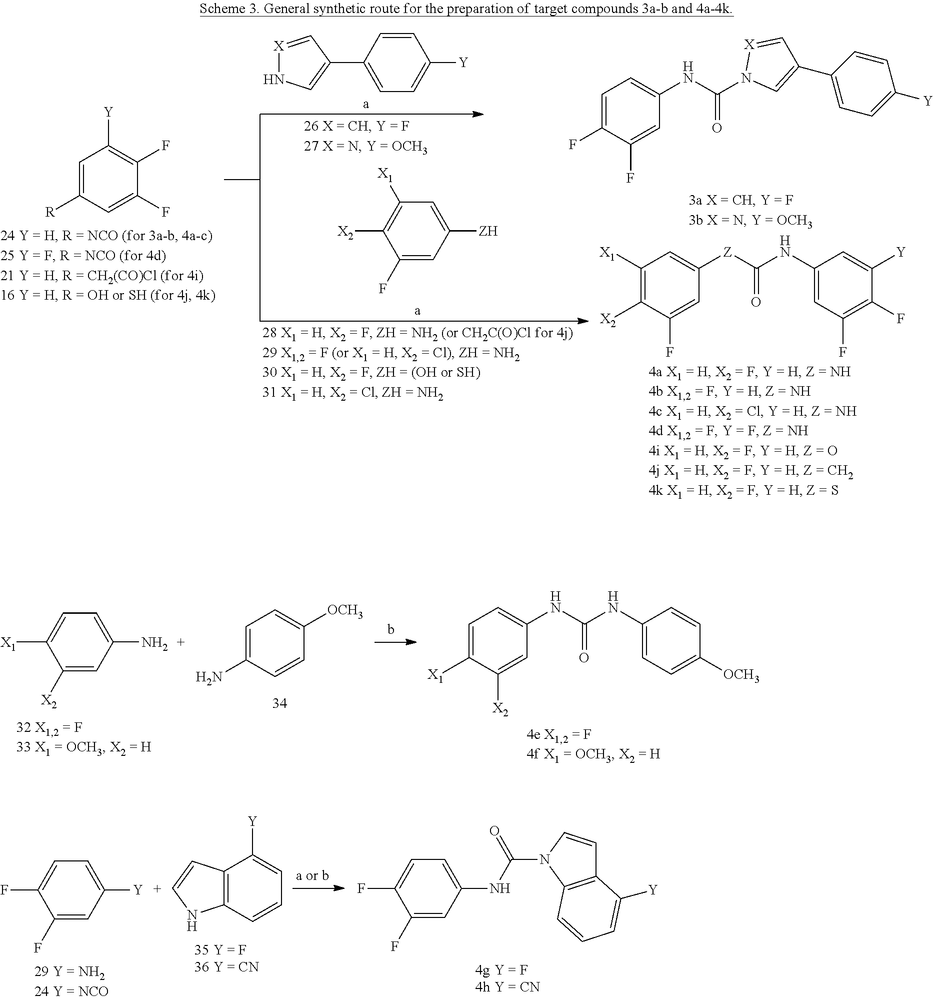

The preparation of modified linker derivates compounds 3a-b and no bridge analogues compounds 4a-h is shown in Scheme 3. The synthetic approach of compounds 3a-b, 4a-d was performed with the isocyanate 24, 25 with corresponding anilines 26˜31 in basic conditions under anhydrous atmosphere. And in using bis(trichloromethyl) carbonate (BTC), two different anilines (29 and/or 32˜35) generated to compounds 4e-g as asymmetric urea typed compounds. The reaction of indole 36 with isocyanate 24 produced urea type compound 4 h. The unsymmetric compounds of carbamate compound 4i, acetamide compound 4j and carbamothioate compound 4k, generated from substituted phenol (or thiol) 30, or acid chloride 21 (for compound 4j) from corresponding carboxylic acid by treating SOCl₂ as shown in Scheme 3.

### Example 4

### Synthesis of Compound Series 5

In Scheme 4, the mediated reaction of bis(trichloromethyl) carbonate (BTC) with compound 37R produced compound 5 as symmetric product with compound 5a as by product. Reagents and conditions: (a) bis(trichloromethyl) carbonate, Et₃N, DCM, rt.

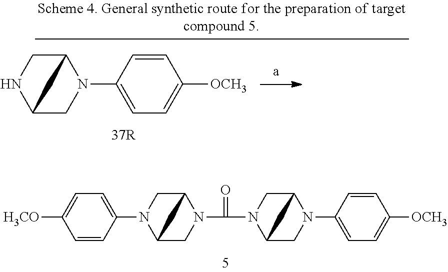 [SMILES: COc1ccc(N2C[C@H]3C[C@@H]2CN3)cc1 ; COc1ccc(N2C[C@H]3C[C@@H]2CN3C(=O)N2C[C@H]3C[C@@H]2CN3c2ccc(OC)cc2)cc1]

### Example 5

### Structure-Activity Relationship (SAR) Exploration of Compound Series 2, 3, 4 and 5

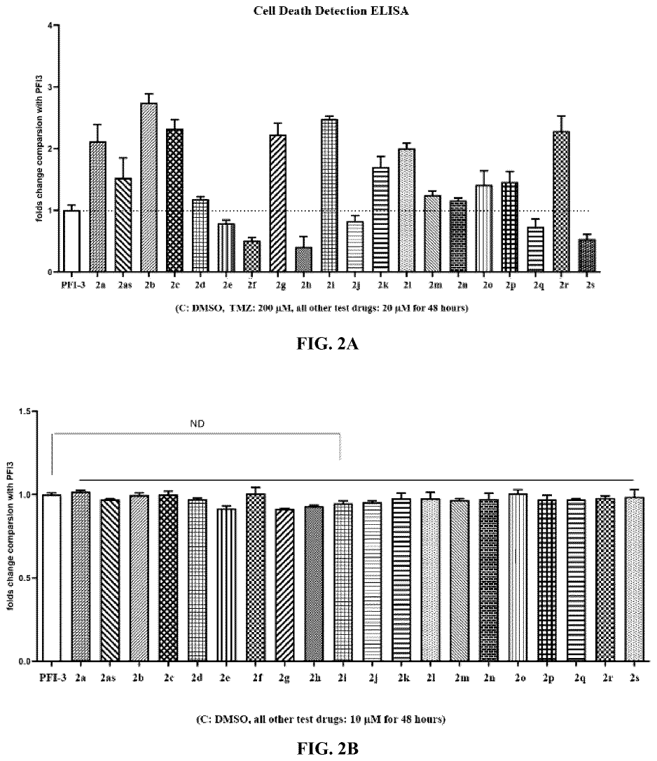

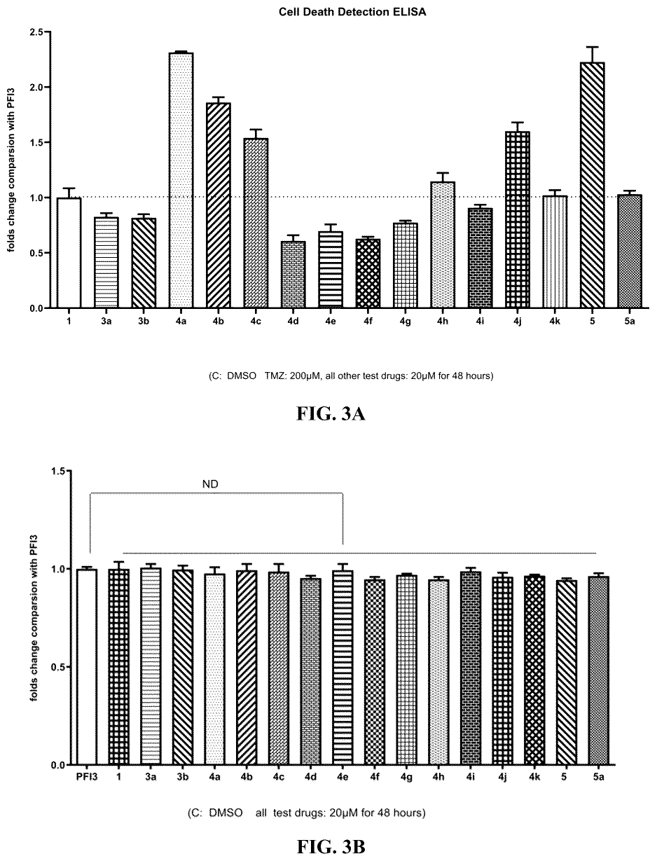

An SAR study was conducted derived from core cyclization of ortho-keto-phenol from the A-ring part of PFI-3 converting to substituted benzene ring (described in FIG. 1) succeeded in developing new group bromodomain inhibitors denoted as new TEDs, so the first analog (2) had better activity than PFI-3 as a bromodomain inhibitor and also following analog (4) was gave insights into what structural modifications in bromodomain inhibitors resulted in more biologically potent analogs (as shown in FIG. 2A and FIG. 3A).

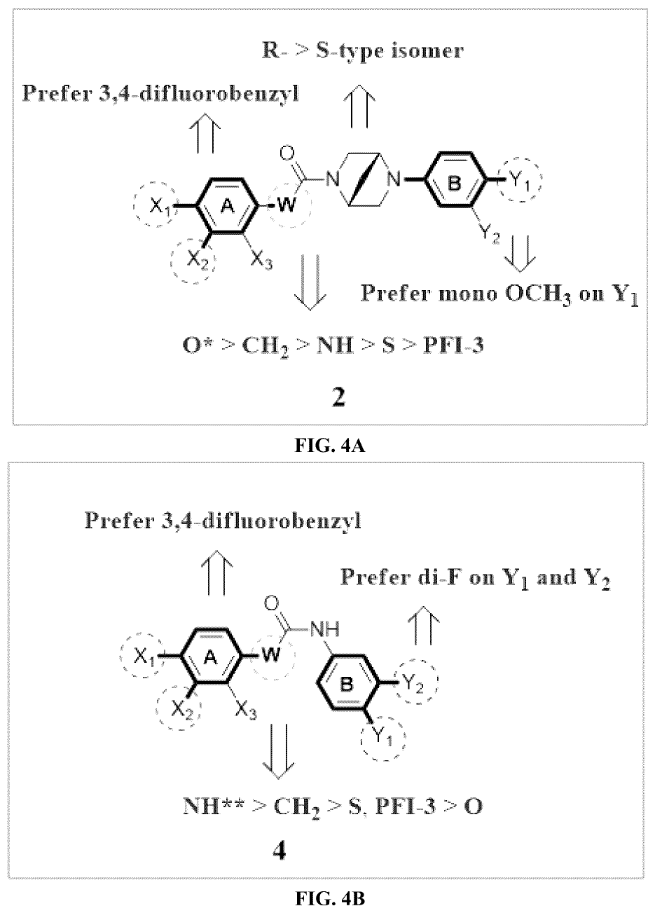

In FIG. 4A and FIG. 4B, an overview of the re-scaffolding studies is presented from PFI-3 to new compound series 2 (FIG. 4A) and compound series 4 (FIG. 4B). The initial re-scaffolding of compound series 2, in which “a benzene moiety” in the ortho-keto-phenol of PFI-3 was replaced with simple molecules, resulting into non-bridge series of compounds 4a-h. The introduction of different bridged series 3 (i.e., compounds 3a and 3b, an unsaturated 5-membered ring) confirmed the requirement of this structural feature in producing new analogs more potent than PFI-3 (shown in FIG. 3A). However, no-A-ring of compound 5 demonstrated showed variable potency in their ability to enhance TMZ-induced GBM cell death as compared to PFI-3 as shown in FIG. 3A. The enhancing activity of series 2, 4 and 5 on TMZ-induced GBM cell death is also independent of the ability of these compounds to induce cell death themselves alone as shown in FIG. 2B and FIG. 3B. In summary, the structure-activity relationship studies of TED series 2, 3, 4 and 5 specified that modification (i.e. A-ring, linker, bridge and B-ring) the suggested that they are indispensable structural motifs that must be retained to enhance TMZ-induced cell death. On the structural modifications, the structural requirement in these new TEDs to sensitize GBM cells to TMZ-induced death as new bromodomain inhibitor scaffolds are as follows:
A-ring modification (W═NH, Y₁═OCH₃ in Series 2): 3,4-di-Fluorophenyl (for compound 2a)>4-fluorophenyl (for compound 2k)>aliphatic (for compound 2q)>4-cyano or 4-trifluoromethylphenyl (for compounds 2o, 2p)>4-methoxyphenyl (for compound 2n)>>2,4-difluorophenyl (for compound 2m)>PFI-3 (1)>2-chlorophenyl-(for compound 2s), 3-chlorobenzo-(for compound 2h).
A-ring modification (W═O, Y₁═OCH₃ in Series 2): 3,4-di-Fluorophenyl (compound 2b)>unsubstituted phenyl (compound 2i)>cyclohexyl (compound 2r)>>PFI-3 (1)
Linker modification (W═NH, O, CH₂ and S in Series 2): Carbamate compound 2b (W═O)>methylene-amide compound 2c (W═CH₂)>urea compound 2a (W═NH)>carbamothioate compound 2d (W═S)>PFI-3 (1).
Linker modification (W═NH, O, CH₂ and S in Series 4): Urea compound 4a (W═NH)>methylene-amide compound 4j (W═CH₂)>carbamothioate compound 4k (W═S), PFI-3 (1)>carbamate compound 4i (W═0).
Bridge modification (bridged name as “diazabicyclo[2.2.1]heptane” in 2 and 5-membered ring in 3: R-type in 2 (compound 2a)>S-type in 2 (compound 2as)>PFI-3 (1), 5-membered linker 2 (pyrrole compound 3b, pyrazole compound 3a).
A-ring & B-ring modification (for no-bridge urea Series 4): 4 Fluoro substituents in A & B-ring (for 4a)>5 fluoro substituents in A & B-ring (for compound 4b)>3 fluoro substituents in A & B-ring (for compound 4c)>4-cyanoindole in B-ring (for 4 h) PFI-3 (1)>mono-fluoro or methoxy substituents (for compounds 4e, 4f).

Dimeric compound 5 (i.e. no-A-ring) presented very strong activity (two times stronger than PFI-3 as shown in FIG. 3A), and it seems to be influenced by “a symmetric bridged with B-ring” to be active inhibition functions fitted well to the target bromodomain binding site.

For the new scaffolded analogues of compound series 2, 3, 4 and 5, the structural activity relationship (SAR) of bromodomain inhibitors for the treatment of GBM was explored as demonstrated in FIGS. 2A, 2B, 3A, and 3B. Most mono- or multi-substitution on the A or B ring markedly increased GBM sensitization to the cell-death inducing activity of TMZ as much better or compared to PFI-3 (compounds 2, 3, 4 and 5). Primarily compounds of series 2 (compounds 2a, 2b, 2c, 2d, 2g, 2i, 2k, 21, and 2r), 4 (compounds 4a, 4b, 4c, 4g and 4j) and compound 5 were found to be superior to PFI-3 in enhancing the activity of TMZ to induce GBM cell death, as summarized in FIGS. 4A and 4B. Further refinements of the structure by modifying the 5-membered ring in compounds 3a and 3b compromised their activity in GBM cells to the TMZ induced cell relative to PFI-3. Treatment of GBM cells with dimeric compound 5, which had no A-ring of re-scaffolding structure 2, resulted in two times increased activity to the TMZ induced cell death than PFI-3 (compound 1). Moreover, removal of the bridge portion in compounds (urea 4a and carbamate 4b) resulted in compounds with relatively high sensitivity to the death-inducing activity with TMZ.

### Example 5

### Calculated Properties of PFI-3 and Compounds Series 2, 3, 4 and 5

Compounds of series 2, 3, 4, and 5 display high gastrointestinal (GI) absorption, penetration in the blood-brain barrier (BBB) and optimized physical properties in computer simulation shown in Table 1.

The constituents of new modified PFI-3 analogues of compound series 2, 3, 4 and 5 were examined by a computer-aided prediction known as Swiss ADME programs (www.swissadme.ch, Molecular Modeling Group—Swiss Institute of Bioinformatics, Lausanne, CH) to predict a drug option. The synthesized compound series 2, 3, 4 and 5 calculated and expected physical properties, such as ADME (Absorption, Distribution, Metabolism, and Excretion) and other drug types to use the pharmaceutical aids as shown in Table 1.

TABLE 1
Summarized Physicochemical, Pharmacokinetic, and Drug likeness properties of
compounds 2a-c, 2as, 3b, 4a, 4i-k, and 5a by Swiss ADME web tool

| | | | | | | | Pharmacokinetic | | | |
|---|---|---|---|---|---|---|---|---|---|---|
| | | | | | | | parameters | | Drug likeness properties | |
| | Physicochemical Parameters | | | | | | GI | BBB | Lipinski's | Bio |
| ID | HA | RB | HBA | HBD | FC | MLOGP | absorption | permeation | Rules of Five | availability |
| 2a | 26 | 5 | 4 | 1 | 0.32 | 3.38 | High | Yes | Yes | 0.55 |
| | | | | | | | | | 0 violation | |
| 2aS | 26 | 5 | 4 | 1 | 0.32 | 3.38 | High | Yes | Yes | 0.55 |
| | | | | | | | | | 0 violation | |
| 2b | 26 | 5 | 5 | 0 | 0.32 | 3.38 | High | Yes | Yes | 0.55 |
| | | | | | | | | | 0 violation | |
| 2c | 26 | 5 | 4 | 0 | 0.35 | 3.36 | High | Yes | Yes | 0.55 |
| | | | | | | | | | 0 violation | |
| 3a | 23 | 4 | 1 | 4 | 0 | 4.53 | High | Yes | 1 violation | 0.55 |
| 3b | 24 | 5 | 1 | 5 | 0.06 | 3.55 | High | Yes | Yes | 0.55 |
| | | | | | | | | | 0 violation | |
| 4a | 20 | 4 | 5 | 2 | 0 | 4.53 | High | Yes | 1 violation | 0.55 |
| 4i | 20 | 4 | 6 | 1 | 0 | 4.53 | High | Yes | 1 violation | 0.55 |
| 4j | 20 | 4 | 5 | 1 | 0.07 | 4.58 | High | Yes | 1 violation | 0.55 |
| 4k | 20 | 4 | 5 | 1 | 0 | 5.01 | High | Yes | 1 violation | 0.55 |
| 5 | 32 | 6 | 3 | 0 | 0.48 | 2.58 | High | Yes | Yes | 0.55 |
| | | | | | | | | | 0 violation | |

HA: Number of heavy atoms,
RB: Number of rotatable bonds,
HBA: Number of H-bond acceptors;
HBD: Number of H-bond donors;
FC: Fraction Csp3;
MLOGP: LogP_{o/w} Topological method,
GI absorption: Gastrointestinal absorption,
BBB permeation: Blood-Brain Barrier permeation.

Table 1 shows the key factors of physical properties for drug-likeness such as physicochemical, pharmaceutical, and drug-likeness properties of modified PFI-3 analogues selected compounds of series 2 through 5, especially high GI absorption and penetrable properties on BBB with high bioavailability matched with Lipinski's Rules of Five.

### Example 6

### General Methods of Preparation for the Synthesis of Compound Series 2, 3, 4 and 5

### Method A

A 100 mL, oven-dried, two-necked, round-bottomed flask is charged with a teflon-coated magnetic oval stir bar and coupled with a 50 mL dropping funnel. Both the dropping funnel and the round-bottomed flask are sealed with a rubber septum. Under argon atmosphere, to a solution of triphosgene (520 mg, 1.75 mmol) in 10 mL of dry THF, compound 15S (325 mg, 1.6 mmol) in 3 mL of THF was added slowly at 0° C. The resulting mixture was stirred at the same temperature for 10 min and allowed to stir at room temperature for another 30 min. After completion of reaction, the solution of substituted aniline (1.48 mmol) with 1 mL of Et₃N was added to the mixture and stirred overnight at room temperature. The solution was concentrated under reduced pressure and poured into EtOAc, washed with sat. NaHCO₃ solution, water, dried over anhydrous MgSO₄ and concentrated under reduced pressure to purify by silica gel chromatography (EtOAc/n-hexane=1:1) or (hexane/Acetone=3:1, v/v) to afford the designed compound.

### Method B

Under nitrogen atmosphere, to a solution of isocyanate (23, 24 or 25, 2.2 mmol) in 10 mL of dry DCM, aniline (15R, 18R, 26 or 27, 2 mmol) in 3 mL of DCM and triethylamine (0.3 mL) was added slowly at 0° C. The resulting mixture was stirred at the same temperature for 10 min and allowed to stir at room temperature for another 30 min. After completion of reaction, the reaction mixture was added to crushed ice and extracted with DCM. The organic layer was dried over anhydrous MgSO₄ and concentrated under reduced pressure to purify by a silica gel chromatography (EtOAc/n-hexane=1:2) or (acetone/hexane=1:3, v:v) to afford the target compound.

### Method C

To a solution of (1R,4R)-tert-butyl 2,5-diazabicyclo[2.2.1]heptane-2carboxylate 12R, 5 mmol) in anhydrous toluene (30 mL) was added substituted bromobenzene (13, 10 mmol), sodium tert-butoxide (5 mol), Pd(OAc)₂ (0.25 mmol), and (R)(+)-2,2′-bis(diphenylphosphino)-1,1′-binaphthalene (BINAP, 0.25 mmol) at room temperature under the argon atmosphere. The resulting reaction mixture was heated at reflux for 4-5 h under argon atmosphere. After the end of the reaction was monitored by TLC, the reaction was quenched by water, extracted with ethyl acetate. The organic layer was dried with anhydrous MgSO₄, filtered, concentrated under reduced pressure, and purified by column chromatography using hexanes and ethyl acetate (1:1, v/v) as eluent to afford the desired compound.

### Method D

The solution of compound 14R (40.36 mmol) was dissolved in anhydrous ethanol (30 mL) in a 100-ml round-bottomed flask. To this, 5 mL of acetyl chloride was drop-wisely added at ice-water bath and was stirred overnight at room temperature under argon conditions. The reaction was monitored by TLC using ethyl acetate and hexane (2:3, v/v) system. Stirring was continued until TLC indicated the completion of reaction. The solution was reduced off under reduced pressure. The solvent was removed completely under vacuum to get compound 15R.

### Synthesis of Compounds 3a and 3b

A 100 mL, oven-dried, two-necked, round-bottomed flask is charged with a Teflon-coated magnetic oval stir bar and coupled with a 50 mL dropping funnel. Both the dropping funnel and the round-bottomed flask are sealed with a rubber septum. Under nitrogen atmosphere, to a solution of 1,2-difluoro-4-isocyanatobenzene (compound 24, 320 mg, 2.06 mmol) in 10 mL of dry DCM, 3-(4-fluorophenyl)-1H-pyrrole (compound 26, for compound 3a) or 4-(4-methoxyphenyl)-1H-pyrazole (compound 27, for compound 3b) (1.6 mmol) in 3 mL of DCM and triethylamine (0.2 mL) was added slowly at 0° C. The resulting mixture was stirred at the same temperature for 10 min and allowed to stir at room temperature for another 30 min. After completion of reaction, the reaction mixture was added to crushed ice and extracted with DCM. The organic layer was dried over anhydrous MgSO₄ and concentrated under reduced pressure to purify by a silica gel chromatography (Acetone/hexane=1:4, v/v) to afford the target compounds.

### Example 7

### Preparation of (1R,4R)-tert-Butyl 5-(pyridin-2-yl)-2,5-diazabicyclo[2.2.1]heptane-2-carboxylate (Compound 14Ra, Z═N, Y_{1, 2}═H in Scheme 1)

Prepared by Method C as described in Example 6.

MS (ESI) m/z 276.20 [M+H]⁺; LCMS (ESI) m/z calcd for C₁₅H₂₂N₃O₂: 276.1712 [M+H]⁺, Found: 276.1716 [M+H]⁺. ¹H NMR (400 MHz, DMSO-d₆) δ 8.06 (d, J=4.8 Hz, 1H, ArH), 7.51-7.47 (m, 1H, ArH), 6.60-6.57 (m, 1H, ArH), 6.54-6.51 (m, 1H, ArH), 4.77 (d, J=8.8 Hz, 1H, CH), 3.51-3.45 (m, 1H, CH), 3.44-3.30 (m, 2H, CH), 3.26-3.33 (m, 1H, CH), 3.18-3.15 (m, 1H, CH), 1.90-1.87 (m, 2H, CH), 1.39 (s, 6H, 2×CH₃), 1.34 (s, 3H, CH₃).

### Example 8

### Preparation of (1R,4R)-2-(pyridin-2-yl)-2,5-diazabicyclo[2.2.1]heptane (Compound 15Ra, Z═N, Y_{1,2}═H in Scheme 1)

Prepared by Method D as described in Example 6.

MS (ESI) m/z 176.06 [M+H]⁺, 198.09 [M+Na]⁺. ¹H NMR (400 MHz, DMSO-d₆) δ 8.13 (d, J=4.8 Hz, 1H, ArH), 7.48-7.43 (m, 1H, ArH), 6.63-6.58 (m, 1H, ArH), 6.37-6.32 (m, 1H, ArH), 5.03 (d, J=7.8 Hz, 1H, CH), 4.54-4.50 (m, 1H, CH), 3.68-3.65 (m, 1H, CH), 3.61-3.49 (m, 2H, CH), 3.44-3.38 (m, 1H, CH), 2.12-1.91 (m, 2H, CH).

### Example 9

### Preparation of (1R,4R)-tert-Butyl 5-(4-methoxyphenyl)-2,5-diazabicyclo[2.2.1]heptane-2-carboxylate (Compound 14Rb, Z═CH, Y₁═OCH₃, Y₂═H in Scheme 1)

Prepared by Method C as described in Example 6.

White solid. Yield (75%). UV max: 249.45, 315.45 nm. Purity (LC: t_{R} 3.60 min): 97.54%. MS (ESI) m/z 305.15 [M+H]⁺. LCMS (ESI) m/z calcd for C₁₇H₂₄N₂O₃: 305.1865 [M+H]⁺, Found: 305.1853 [M+H]⁺. ¹H NMR (400 MHz, CDCl₃) δ 6.84 (t, J=8.4 Hz, 2H, ArH), 6.52 (dd, J=8.4, 6.0 Hz, 2H, ArH), 4.50 (d, J=58.4 Hz, 1H), 4.31 (s, 1H), 3.76 (s, 3H, OCH3), 3.58 (m, 1H), 3.49 (dd, J=31.6, 10.0 Hz, 1H), 3.35 (t, J=12.8 Hz, 1H), 3.14 (dd, J=38.0, 8.4 Hz, 1H), 1.98 (m, 1H), 1.90 (m, 1H), 1.44 (s, 3H), 1.40 (s, 6H).

### Example 10

### Preparation of (1R,4R)-tert-Butyl 5-(3,4-dimethoxyphenyl)-2,5-diazabicyclo[2.2.1]heptane-2-carboxylate (Compound 14Rc, Z═CH, Y_{1, 2}═OCH₃, in Scheme 1)

Prepared by Method C as described in Example 6.

White-off solid. Yield 70%. MS (ESI) m/z 335.12 [M+H]⁺; LCMS (ESI) m/z calcd for C₁₈H₂₆N₂O₄: 335.1971 [M+H]⁺, Found: 335.1989 [M+H]⁺. ¹H NMR (400 MHz, DMSO-d₆) δ 6.78 (d, J=8.4 Hz, 1H, ArH), 6.22 (d, J=2.4 Hz, 1H, ArH), 6.06 (dd, J=8.4, 2.4 Hz, 1H), 4.41 (bs, 1H, NH), 4.39 (m, 1H), 3.73 (s, 3H, OCH₃), 3.63 (s, 3H, OCH₃), 3.51 (m, 1H), 3.28 (m, 1H), 3.23 (m, 1H), 2.90 (t, J=9.8 Hz, 1H), 1.88 (m, 1H), 1.87 (m, 1H), 1.40 (s, 6H), 1.32 (s, 3H). ¹³C NMR (100 MHz, DMSO-d₆) δ 153.92, 150.60, 142.72, 140.80, 114.87, 104.14, 99.18, 79.07, 58.09, 57.54, 56.96, 56.53, 55.91, 51.21, 37.55, 28.58.

### Example 11

### Preparation of (1R,4R)-2-(3,4-Dimethoxyphenyl)-2,5-diazabicyclo[2.2.1]heptane (Compound 15Rc)

Prepared by Method D as described in Example 6.

Light brown solid. Yield 95%. LCMS (ESI) m/z calcd for C₁₃H₁₈N₂O₂: 235.1447 Found: 235.1502 [M+H]⁺. ¹H NMR (400 MHz, DMSO-d₆) δ 9.80 (bs, 1H, NH), 9.02 (bs, 1H, NH), 6.82 (d, J=8.8 Hz, 1H, ArH), 6.29 (d, J=2.0 Hz, 1H, ArH), 6.12 (dd, J=8.8, 2.0 Hz, 1H), 3.72 (s, 3H, OCH₃), 3.78 (m, 1H), 3.64 (s, 3H, OCH₃), 3.64-3.52 (m, 2H), 3.26 (d, J=10.0 Hz, 1H), 3.17-3.12 (m, 2H), 2.08 (d, J=10.4 Hz), 1.93 (d, J=10.4 Hz, 1H). ¹³C NMR (100 MHz, DMSO-d₆) δ 150.41, 141.58, 141.52, 114.67, 104.88, 99.81, 57.65, 56.88, 56.06, 52.89, 48.69, 35.95.

### Example 12

### Preparation of (1S,4S)—N-(3,4-Difluorophenyl)-5-(4-methoxyphenyl)-2,5-diazabicyclo[2.2.1]heptane-2-carboxamide (Compound 2as)

Prepared by Methods A & B as described in Example 6.

White off solid. Yield 80%. UV max: 195.45, 230.45. Purity (LC, t_{R} 3.10 min) 97.09%. MS (ESI) m/z 360.15 [M+H]⁺; 358.23 [M−H]⁻. LCMS (ESI) m/z calcd for C₁₉H₁₉F₂N₃O₂: 360.1524 [M+H]⁺. Found: 360.1525 [M+H]⁺; ¹H NMR (400 MHz, DMSO-d₆) δ 8.43 (bs, 1H, NH), 7.65-7.59 (m, 1H, ArH), 7.03 (q, J=9.60 Hz, ArH), 7.21 (m, 1H, ArH), 6.79 (d, J=8.8 Hz, 2H, ArH), 6.56 (d, J=8.8 Hz, 2H, ArH), 4.67 (s, 1H), 4.49 (s, 1H), 4.49 (s, 1H), 3.64 (s, 3H, OCH₃), 3.55 (d, J=8.0 Hz, 1H), 3.39 (s, 2H), 2.94 (d, J=8.8 Hz, 1H), 1.98 (d, J=10.8 Hz, 1H), 1.91 (d, J=8.8 Hz, 1H). ¹⁹F NMR (400 MHz, DMSO-d₆) δ −136.52, −145.34.

### Example 13

### Preparation of (1R,4R)-3,4-Difluorophenyl 5-(4-methoxyphenyl)-2,5-diazabicyclo[2.2.1]heptane-2-carboxylate (Compound 2b)

Prepared by Method A as described in Example 6.

Light yellowish solid. Yield 51%. MS (ESI) m/z 361.12 [M+H]⁺. UV max: 240.45, 315.45 nm. Purity (LC, t_{R} 3.64 min) 98.77%. LCMS (ESI) m/z calcd for C₁₉H₁₈F₂N₂O₃: 361.1364 [M+H]⁺, Found: 361.1353 [M+H]⁺. ¹H NMR (400 MHz, DMSO-d₆) δ 7.46-7.40 (m, 1H, ArH), 7.38-7.32 (m, 1H, ArH), 7.05-6.95 (m, 1H, ArH), 6.82 (d, J=8.4 Hz, 1H), 6.62 (d, J=8.4 Hz, 1H), 4.69 (s, 1H), 4.55 (d, J=6.0 Hz, 1H), 3.65 (s, 3H, OCH₃), 3.62 (m, 2H), 3.58-3.32 (m, 3H), 3.16 (d, J=8.8 Hz, 1H), 2.02 (m, 1H), 1.98 (m, 1H). ¹⁹F NMR (400 MHz, DMSO-d₆) δ −136.50, −142.66

### Example 14

### Preparation of 2-(3,4-Difluorophenyl)-1-((1R,4R)-5-(4-methoxyphenyl)-2,5-diazabicyclo[2.2.1]heptan-2-yl)ethenone (Compound 2c)

Under nitrogen atmosphere, thionyl chloride (18.4 mL, 0.252 mol) was added dropwise to a cooled solution (less than 4° C.) of 2-(3,4-difluorophenyl)acetic acid (206 mg, 1.2 mmol) in 7 mL of THF under an argon atmosphere. The resulting mixture was stirred for 3 h under the same condition. The solution was drop wisely added to a solution of compound 15R (204 mg, 1 mmol) in DCM/Pyridine (2 mL/5 mL) and stirred overnight at rt. The solvent was removed under reduced pressure and diluted with EtOAc (20 mL), washed with H₂O, 5% HCl solution and brine (300 mL). The organic layer was dried over anhydrous MgSO₄ and concentrated under reduced pressure to give a crude solid which was purified from column chromatography using acetone/hexane (1/4, v/v) to give a solid.

Light yellowish solid. Yield 53%. MS (ESI) m/z 359.12 [M+H]⁺. UV max: 190.45, 209.45, 249.45 nm. Purity (LC, t_{R} 3.06 min) 98.73%. LCMS (ESI) m/z calcd for C₂₀H₂₀F₂N₂O₂: 359.1571 [M+H]⁺. Found: 359.1578 [M+H]⁺. ¹H NMR (400 MHz, DMSO-d₆) δ 7.35 (m, 1H, ArH), 7.24 (m, 1H, ArH), 7.15 (m, 1H, ArH), 6.78 (m, 2H, ArH), 6.52 (m, 2H, ArH), 4.78 (d, J=25.6 Hz, 1H), 4.51 (d, J=37.6 Hz, 1H), 3.76 (d, J=15.6 Hz, 1H), 3.65 (s, 3H, OCH₃), 3.55 (m, 1H), 3.51-3.44 (m, 2H), 3.29 (s, 1H), 2.87 (t, J=10.2 Hz, 1H), 2.01 (d, J=9.6 Hz, 1H), 1.93 (d, J=9.6 Hz, 1H). ¹⁹F NMR (400 MHz, DMSO-d₆) δ −139.59, −142.26.

### Example 15

### Preparation of (1R,4R)—S-(3,4-Difluorophenyl) 5-(4-methoxyphenyl)-2,5-diazabicyclo[2.2.1]heptane-2-carbothioate (Compound 2d)

Under argon atmosphere, to a solution of triphosgene (445 mg, 1.5 mmol) in 10 mL of anhydrous THF, 3,4-difluorothiophenol (219 mg, 1.5 mmol) in 3 mL of THF was added slowly at 0° C. to the solution and triethylamine (0.28 mL, 2 mmol) was dropwise added in the solution. The resulting mixture was stirred at the same temperature for 10 min and allowed to heat to reflux for 30 min. After cooling the flask, the solution of 3,4-42R (306 mg, 1.5 mmol) in 2 mL anhydrous THF was added to the mixture and stirred overnight at room temperature. The reaction mixture was concentrated under reduced pressure and then dissolved into 30 mL of ethyl acetate, washed with water (30 mL), brine (30 mL). The organic layer was dried over anhydrous MgSO₄, and concentrated under reduced pressure, purified with flash column chromatography using EtOAc/Hex (1:2, v/v) to give a white solid. Yield 53%. MS (ESI) m/z 377.10 [M+H]⁺. UV max: 192.45, 248.45, 249.45 nm. Purity (LC, t_{R} 3.53 min): 98.39%. LCMS (ESI) m/z calcd for C₁₉H₁₈F₂N₂O₂S: 377.1135 [M+H]⁺. Found: 359.1136 [M+H]⁺. ¹H NMR (400 MHz, DMSO-d₆) δ7.60-7.57 (m, 1H, ArH), 7.47-7.43 (m, 1H, ArH), 7.29 (m, 1H, ArH), 6.78 (m, 2H, ArH), 6.52 (m, 2H, ArH), 4.78 (m, 1H), 4.56 (s, 1H), 3.66 (s, 3H, OCH₃), 3.54 (t, J=9.2 Hz, 1H), 3.39 (m, 2H), 3.05 (d, J=9.2 Hz, 1H), 2.04 (m, 1H), 1.98 (m, 1H). ¹⁹F NMR (400 MHz, DMSO-d₆) δ −13.82, −136.08.

### Example 16

### Preparation of (1R,4R)-5-(3,4-Dimethoxyphenyl)-N-(4-fluorophenyl)-2,5-diazabicyclo[2.2.1]heptane-2-carboxamide (Compound 2e)

Prepared by Method A as described in Example 6.

White-off solid. Yield 59%. Purity: (LC, tₘᵢₙ=3.08) 99.46%. UV max: 210.45, 238.45, 307.45.

MS (ESI) m/z 372.23 [M+H]⁺; 370.29 [M−H]⁻. LCMS (ESI) m/z calcd for C₂₀H₂₂FN₃O₃: 370.1567 [M−H]⁻, Found: 370.1573 [M−H]⁻. ¹H NMR (400 MHz, DMSO-d₆) δ 8.26 (bs, 1H, NH), 7.45 (m, 2H, ArH), 7.02 (dt, J=8.8, 2.0 Hz, 2H, ArH), 6.79 (d, J=8.8 Hz, 1H), 6.24 (d, J=2.4 Hz, 1H), 6.08 (dd, J=8.8, 2.4 Hz, 1H), 4.68 (s, 1H), 4.52 (s, 1H), 3.73 (s, 3H, OCH₃), 3.62 (s, 3H, OCH₃), 3.55 (dd, J=8.8, 1.6 Hz, 1H), 3.41 (s, 2H), 3.01 (d, J=8.8 Hz, 1H), 1.96 (d, J=9.2 Hz, 1H), 1.91 (d, J=9.2 Hz, 1H); ¹⁹F NMR (400 MHz, DMSO-d₆) δ −121.36. ¹³C NMR (100 MHz, DMSO-d₆) δ 152.29 (d, J_{F}=370 Hz), 150.14 (C═O), 150.44, 142.78, 140.80, 137.08, 121.39 (d, J_{F}=8.0 Hz), 115.31 (d, J_{F}=22.0 Hz), 114.92, 104.21, 99.23, 57.88, 57.39, 56.99, 56.64, 55.87, 51.25, 37.30.

### Example 17

### Preparation of (1R,4R)—N-(3,4-Difluorophenyl)-5-(3,4-dimethoxyphenyl)-2,5-diazabicyclo[2.2.1]heptane-2-carboxamide (Compound 2f)

Prepared by Method A as described in Example 6.

Light brown solid. Yield 72%. Purity: (LC, tₘᵢₙ=3.21) 99.27%. UVmax: 210.45, 237.45, 309.45. MS (ESI) m/z 390.19 [M+H]⁺; 388.30 [M−H]⁻. LCMS (ESI) m/z calcd for C₂₀H₂₁F₂N₃O₃: 388.1473 [M−H]⁻, Found: 388.1482 [M−H]⁻. ¹H NMR (400 MHz, DMSO-d₆) δ 8.43 (bs, 1H, NH), 7.63 (m, 1H, ArH), 7.27 (q, J=9.2 Hz, 1H, ArH), 7.21 (m, 1H, ArH), 6.78 (d, J=8.8 Hz, 1H), 6.24 (d, J=2.4 Hz, 1H), 6.09 (dd, J=8.8, 2.4 Hz, 1H), 4.69 (s, 1H), 4.53 (s, 1H), 3.73 (s, 3H, OCH₃), 3.62 (s, 3H, OCH₃), 3.56 (dd, J=8.8, 1.6 Hz, 1H), 3.42 (s, 2H), 3.01 (d, J=8.8 Hz, 1H), 1.97 (d, J=9.6 Hz, 1H), 1.91 (d, J=9.6 Hz, 1H). ¹⁹F NMR (400 MHz, DMSO-d₆) δ −138.12, −147.64. ¹³C NMR (100 MHz, DMSO-d₆) δ 153.79 (C═O), 150.44, 148.06 (d, J_{F}=13.0 Hz), 142.73, 140.84, 138.06, 148.06 (d, J_{F}=9.0 Hz), 117.40 (d, J_{F}=18.0 Hz), 115.42 (d, J_{F}=6.0 Hz), 114.90, 108.34 (d, J_{F}=22.0 Hz), 104.23, 99.26, 57.85, 57.36, 56.98, 56.69, 55.87, 51.25, 37.25.

### Example 18

### Preparation of (1R,4R)—N-(3,4-Difluorophenyl)-5-(pyridin-2-yl)-2,5-diazabicyclo[2.2.1]heptane-2-carboxamide (Compound 2g)

Prepared by Method B as described in Example 6.

Yield 62% as White foam. Purity: (LC, tₘᵢₙ=2.08) 97.61%; UV max: 204.45, 239.45, 319.45; MS (ESI) m/z 329.19 [M−H]⁻; 331.13 [M+H]⁺; LCMS (ESI) m/z calcd for C₁₇H₁₆F₂N₄O: 331.1370 [M+H]⁺, Found: 331.1370 [M+H]⁺; ¹H NMR (400 MHz, DMSO₆) δ 8.48 (bs, 1H, NH), 8.07 (dd, J=3.0, 0.6 Hz, 1H, ArH), 7.68 (dq, J=7.6, 2.4 Hz, 1H, ArH), 7.52-7.48 (m, 1H, ArH), 7.31 (q, J=9.2 Hz, 1H, ArH), 7.24 (m, H, ArH), 6.61 (dd, J=6.4, 5.2 Hz, 1H, ArH), 6.55 (d, J=8.4 Hz, 1H, ArH), 4.86 (s, 1H, CH), 4.76 (s, 1H, CH), 3.54-3.49 (m, 2H, CH), 3.38-3.31 (m, 2H, CH), 2.00-1.96 (m, 2H, CH); ¹³C NMR (100 MHz, DMSO-d₆) δ 157.36 (C═O), 153.92, 148.33, 148.06 (dq, J_{F}=241.0, 13.0 Hz), 138.02 (q, J_{F}=2.0 Hz), 137.74, 117.40 (d, J_{F}=17.0 Hz), 115.55 (q, J_{F}=2.0 Hz), 112.55, 108.42 (d, J_{F}=22.0 Hz), 107.60, 56.76, 56.55, 55.82, 53.06, 37.06; ¹⁹F NMR (400 MHz, DMSO-d₆) δ −138.11, −147.56.

### Example 19

### Preparation of (1R,4R)—N-(7-Chlorobenzo[c][1,2,5]oxadiazol-4-yl)-5-(4-methoxyphenyl)-2,5-diazabicyclo[2.2.1]heptane-2-carboxamide (Compound 2h)

Prepared by Method A as described in Example 6.

Yellow solid. Yield 70%. MS (ESI) m/z 398.27 [M−H]⁻. LCMS (ESI) m/z calcd for C₁₉H₁₈ClN₅O₃: 400.1176 [M+H]⁺, Found: 400.1176 [M+H]⁺; 398.1020 [M−H]⁻ Found: 398.1046 [M−H]⁻. ¹H NMR (400 MHz, DMSO-d₆) δ 7.41 (d, J=8.0 Hz, 1H, ArH), 6.81 (d, J=8.8 Hz, 2H, ArH), 6.79 (bs, 1H, NH), 6.59 (d, J=8.8 Hz, 1H, ArH), 6.28 (d, J=8.0 Hz, 1H, ArH), 4.75 (m, 1H), 4.52 (m, 1H), 3.65 (s, OCH₃, 3H), 3.63-3.54 (m, 1H), 3.05-3.38 (m, 1H), 3.42 (m, 1H), 3.06-2.99 (m, 1H), 2.08-2.05 (m, 1H), 2.01-1.99 (m, 1H). ¹³C NMR (100 MHz, DMSO-d₆) δ 151.59 (C═O), 148.95, 145.50, 137.18, 134.87, 115.24 (2C), 114.68 (2C), 114.33, 114.30, 104.86, 102.14, 62.51, 59.96, 56.94, 56.55, 37.08.

### Example 20

### Preparation of (1R,4R)-Phenyl 5-(4-methoxyphenyl)-2,5-diazabicyclo[2.2.1]heptane-2-carboxylate (Compound 2i)

Prepared by Method A as described in Example 6.

Yield 88%, Pinkish solid, Purity: (LC, tₘᵢₙ=3.35) 97.23%; UV max: 191.45, 249.45; MS (ESI) m/z 325.11 [M+H]⁺; LCMS (ESI) m/z calcd for C₁₉H₂₀N₂O₃: 325.1552 [M+H]⁺, Found: 325.1549 [M+H]⁺; ¹H NMR (400 MHz, DMSO₆) δ7.39-7.32 (m, 2H, ArH), 7.21-7.18 (m, 1H, ArH), 7.13 (d, J=7.6 Hz, 1H, ArH), 7.07 (d, J=7.6 Hz, 1H, ArH), 6.83 (d, J=8.8 Hz, 2H, ArH), 6.60 (d, J=8.8 Hz, 2H, ArH), 4.71 (s, 1H), 4.54 (d, J=3.6 Hz, 1H), 3.67 (s, 3H, OCH₃), 3.13 (d, J=8.8 Hz, 1H), 2.03-1.99 (m, 2H, CH); ¹³C NMR (100 MHz, DMSO-d₆) δ 152.24 (C═O), 151.39, 141.75, 129.70 (2C), 125.71, 122.71 (2C), 122.19 (2C), 115.23 (2C), 114.23, 58.22, 57.54, 56.77, 55.75, 51.41, 37.63.

### Example 21

### Preparation of (1R,4R)-5-(4-Methoxyphenyl)-N-phenyl-2,5-diazabicyclo[2.2.1]heptane-2-carboxamide (Compound 2j)

Prepared by Method B as described in Example 6.

Yield 82%, White solid. Purity: (LC, t min=2.92) 98.59%; UV max: 191.45, 201.45, 241.45; MS (ESI) m/z 322.86 [M=H]⁻; 324.15 [M+H]⁺; LCMS (ESI) m/z calcd for C₁₉H₂₁N₃O₂: 324.1712 [M+H]⁺, Found: 324.1715 [M+H]⁺; ¹H NMR (400 MHz, DMSO₆) δ 8.22 (bs, 1H, NH), 7.45 (d, J=7.8 Hz, 2H, ArH), 7.18 (t, J=7.8 Hz, 2H, ArH), 6.89 (t, J=7.2 Hz, 1H, ArH), 6.81 (d, J=9.2 Hz, 2H, ArH), 6.58 (d, J=9.2 Hz, 2H, ArH), 4.69 (s, 1H), 4.49 (s, 1H), 3.65 (s, 3H, OCH₃), 3.54 (dd, J=9.0, 1.2 Hz, 1H), 3.41 (s, 2H), 2.98 (d, J=9.2 Hz, 1H), 1.99 (d, J=9.6 Hz, 1H), 1.92 (d, J=9.6 Hz, 1H); ¹³C NMR (100 MHz, DMSO-d₆) δ 154.16 (C═O), 151.28, 141.96, 140.76, 128.73 (2C), 122.05, 119.66 (2C), 115.21 (2C), 114.19 (2C), 58.03, 57.36, 56.63, 55.74, 51.04, 37.30.

### Example 22

### Preparation of (1R,4R)—N-(tert-Butyl)-5-(4-methoxyphenyl)-2,5-diazabicyclo[2.2.1]heptane-2-carboxamide (Compound 2k)

Prepared by Method B as described in Example 6.

Yield 50%, off-white solid. Purity: (LC, tₘᵢₙ=2.97) 95.91%; UV max: 199.45, 236.45; MS (ESI) m/z 340.27 [M−H]⁻; 342.11 [M+H]⁺; LCMS (ESI) m/z calcd for C₁₉H₂₀FN₃O₂: 342.1618 [M+H]⁺, Found: 342.1617 [M+H]⁺; ¹H NMR (400 MHz, DMSO₆) δ 8.27 (bs, 1H, NH), 7.47 (m, 2H, ArH), 7.03 (t, J=8.8 Hz, 2H, ArH), 6.81 (d, J=8.8 Hz, 2H, ArH), 6.58 (d, J=8.8 Hz, 2H, ArH), 4.68 (s, 1H, CH), 4.49 (s, 1H, CH), 3.57 (dd, J=9.2, 2.0 Hz, 1H, CH), 3.40 (s, 2H), 2.97 (d, J=8.8 Hz, 1H), 1.99 (d, J=9.6 Hz, 1H), 1.92 (d, J=9.6 Hz, 1H); ¹³C NMR (100 MHz, DMSO-d₆) δ 158.85 (d, J_{F}=237 Hz), 154.16 (C═O), 151.28, 141.94, 137.10 (d, J_{F}=3.0 Hz), 121.38 (d, J_{F}=8.0 Hz), 115.31 (2C), 115.21, 115.09, 114.19 (2C), 58.01, 57.35, 56.62, 55.73, 51.01, 37.29; ¹⁹F NMR (400 MHz, DMSO-d₆) δ −121.86.

### Example 23

### Preparation of (1R,4R)—N-(3,4-Difluorophenyl)-5-(4-fluorophenyl)-2,5-diazabicyclo[2.2.1]heptane-2-carboxamide (Compound 21)

Prepared by Method B as described in Example 6.

Yield 67%, off-white solid. Purity: (LC, tₘᵢₙ=3.27) 96.89%; UV max: 204.45, 240.45; MS (ESI) m/z 346.20 [M−H]⁻; 348.09 [M+H]⁺; LCMS (ESI) m/z calcd for C₁₈H₁₆F₃N₃O: 348.1324 [M+H]⁺, Found: 348.1326 [M+H]⁺; ¹H NMR (400 MHz, DMSO₆) δ 8.49 (bs, 1H, NH), 7.65 (dq, J=7.6, 2.4 Hz, 1H, ArH), 7.26 (q, J=10.4 Hz, 1H, ArH), 7.23 (m, 1H, ArH), 7.02 (t, J=9.2 Hz, 2H, ArH), 6.62 (m, 2H, ArH), 4.72 (s, 1H, CH), 4.57 (s, 1H, CH), 3.58 (dd, J=9.2, 1.6 Hz, 1H, CH), 3.46-3.40 (m, 2H), 3.02 (d, J=8.8 Hz, 1H), 2.00 (d, J=9.2 Hz, 1H), 1.94 (d, J=9.2 Hz, 1H); ¹⁹F NMR (400 MHz, DMSO-d₆) δ −129.38, −138.11, −147.59.

### Example 24

### Preparation of (1R,4R)-2-(4-Fluorophenyl)-2,5-diazabicyclo[2.2.1]heptane (Compound 15Rd, Z ═CH, Y₁═F, Y₂═H in Scheme 1)

Yield 47%. Yellowish foam. Purity: (LC, tₘᵢₙ=3.27) 96.89%; UV max: 204.45, 240.45; MS (ESI) m/z 346.20 [M−H]⁻; 348.09 [M+H]⁺; LCMS (ESI) m/z calcd for C₁₈H₁₆F₃N₃O: 348.1324 [M+H]⁺, Found: 348.1326 [M+H]⁺; ¹H NMR (400 MHz, DMSO₆) δ 8.49 (bs, 1H, NH), 7.65 (dq, J=7.6, 2.4 Hz, 1H, ArH), 7.26 (q, J=9.2 Hz, 1H, ArH), 7.24-7.22 (m, 1H, AH), 7.02 (t, J=6.4 Hz, ArH), 6.64-6.60 (m, 2H, ArH), 4.72 (s, 1H, CH), 4.60 (s, 1H, CH), 3.58 (dd, J=9.2, 1.6 Hz, 1H, CH), 3.46-3.40 (m, 2H), 3.02 (d, J=8.8 Hz, 1H), 2.00 (d, J=9.2 Hz, 1H), 1.95 (d, J=9.2 Hz, 1H); ¹³C NMR (100 MHz, DMSO-d₆) δ 156.05 (C═O), 153.82, 150.46 (dd, J_{F}=240, 13 Hz), 145.95 (dd, J_{F}=238, 13 Hz), 144.18, 138.00 (d, J_{F}=3 Hz), 117.22 (d, J_{F}=18 Hz), 116.05 (2C), 115.83 (2C), 115.46 (q, J_{F}=4 Hz), 113.96 (d, J_{F}=22 Hz), 57.87, 57.34, 56.68, 37.29; ¹⁹F NMR (400 MHz, DMSO-d₆) δ −129.38, −138.11, −147.59.

### Example 25

### Preparation of 1R,4R)—N-(2,4-Difluorophenyl)-5-(4-methoxyphenyl)-2,5-diazabicyclo[2.2.1]heptane-2-carboxamide (Compound 2m)

Prepared by Method B as described in Example 6.

Yield 87%, Pinkish solid. Purity: (LC, tₘᵢₙ=2.97) 99.34%; UV max: 196.45, 231.45; MS (ESI) m/z 358.15 [M−H]⁻; 360.12 [M+H]⁺; LCMS (ESI) m/z calcd for C₁₈H₁₆F₃N₃O: 360.1524 [M+H]⁺, Found: 360.1493 [M+H]⁺; ¹H NMR (400 MHz, DMSO₆) δ 8.49 (bs, 1H, NH), 7.65 (dq, J=7.6, 2.4 Hz, 1H, ArH), 7.26 (q, J=9.2 Hz, 1H, ArH), 7.24-7.22 (m, 1H, AH), 7.02 (t, J=6.4 Hz, ArH), 6.64-6.60 (m, 2H, ArH), 4.72 (s, 1H, CH), 4.60 (s, 1H, CH), 3.58 (dd, J=9.2, 1.6 Hz, 1H, CH), 3.46-3.40 (m, 2H), 3.02 (d, J=8.8 Hz, 1H), 2.00 (d, J=9.2 Hz, 1H), 1.95 (d, J=9.2 Hz, 1H); ¹³C NMR (100 MHz, DMSO-d₆) δ 154.31 (C═O), 151.27, 141.91, 128.15 (d, J_{F}=10 Hz), 124.08 (dd, J_{F}=12.0, 3.0 Hz), 115.21 (2C), 114.14 (2C), 111.24 (dd, J_{F}=22.0, 3.0 Hz), 104.36 (t, J_{F}=26 Hz), 57.28, 56.83, 55.73, 51.05, 37.39; ¹⁹F NMR (400 MHz, DMSO-d₆) δ −115.56, −117.97.

### Example 26

### Preparation of (1R,4R)—N,S-bis(4-Methoxyphenyl)-2,5-diazabicyclo[2.2.1]heptane-2-carboxamide (Compound 2n)

Prepared by Method B as described in Example 6.

Yield 60%. off-white solid. Purity: (LC, tₘᵢₙ=2.86) 97.29%; UV max: 199.45, 244.45; MS (ESI) m/z 352.80 [M−H]⁻; 354.17 [M+H]⁺; LCMS (ESI) m/z calcd for C₂₀H₂₃N₃O₃: 360.1524 [M+H]⁺, Found: 354.1818 [M+H]⁺; ¹H NMR (400 MHz, DMSO₆) δ 8.07 (bs, 1H, NH), 7.65 (d, J=9.2 Hz, 2H, ArH), 6.80 (d, J=9.2 Hz, 1H, ArH), 6.77 (d, J=9.2 Hz, 1H, ArH), 6.58 (d, J=9.2 Hz, 1H, ArH), 4.66 (s, 1H, CH), 4.48 (s, 1H, CH), 3.68 (s, 3H, OCH₃), 3.65 (s, 3H, OCH₃), 3.55 (dd, J=9.0, 1.6 Hz, 1H, CH), 3.38 (m, 2H), 2.96 (d, J=8.8 Hz, 1H), 1.97 (d, J=8.8 Hz, 1H), 1.91 (d, J=9.2 Hz, 1H); ¹³C NMR (100 MHz, DMSO-d₆) δ 154.74 (C═O), 154.40, 151.26, 141.97, 133.74, 121.57 (2C), 115.21, 114.17, 113.90 (2C), 58.02, 57.38, 56.55, 55.74, 55.51, 50.97, 37.32.

### Example 27

### Preparation of (1R,4R)—N-(4-Cyanophenyl)-5-(4-methoxyphenyl)-2,5-diazabicyclo[2.2.1]heptane-2-carboxamide (Compound 20)

Prepared by Method B as described in Example 6.

Yield 90.0%. off-white solid. Purity: (LC, t min=2.95) 98.71%; UV max: 199.45, 268.45; MS (ESI) m/z 347.24 [M−H]⁻; 349.14 [M+H]⁺; LCMS (ESI) m/z calcd for C₂₀H₂₀N₄O₂: 349.1665 [M+H]⁺, Found: 349.1663 [M+H]⁺; ¹H NMR (400 MHz, DMSO₆) δ 8.71 (bs, 1H, NH), 7.69 (d, J=8.8 Hz, 2H, ArH), 7.64 (d, J=8.8 Hz, 2H, ArH), 6.81 (d, J=8.8 Hz, 2H, ArH), 6.59 (d, J=8.8 Hz, 2H, ArH), 4.73 (s, 1H, CH), 4.51 (s, 1H, CH), 3.65 (s, 3H, OCH₃), 3.57 (dd, J=8.8, 1.6 Hz, 1H, CH), 3.37 (m, 2H), 2.98 (d, J=9.2 Hz, 1H), 1.99 (d, J=7.6 Hz, 1H), 1.93 (d, J=7.6 Hz, 1H); ¹³C NMR (100 MHz, DMSO-d₆) δ 153.40 (C═O), 151.34, 145.39, 141.86, 133.29 (2C), 119.87, 119.10 (2C), 115.21 (2C), 114.22 (2C), 103.33, 57.99, 57.28, 56.83, 55.73, 51.14, 37.22.

### Example 28

### Preparation of (1R,4R)-5-(4-Methoxyphenyl)-N-(4-(trifluoromethyl)phenyl)-2,5-diazabicyclo[2.2.1]heptane-2-carboxamide (Compound 2p)

Prepared by Method B as described in Example 6.

Yield 89%. off-white foam. Purity: (LC, tₘᵢₙ=3.36) 98.47%; UV max: 204.45, 251.45; MS (ESI) m/z 390.13 [M−H]⁻; 392.13 [M+H]⁺; LCMS (ESI) m/z calcd for C₂₀H₂₀F₃N₃O₂: 392.1586 [M+H]⁺, Found: 392.1597 [M+H]⁺; ¹H NMR (400 MHz, DMSO₆) δ 8.63 (bs, 1H, NH), 7.71 (d, J=8.8 Hz, 2H, ArH), 7.57 (d, J=8.8 Hz, 2H, ArH), 6.82 (d, J=9.2 Hz, 2H, ArH), 6.59 (d, J=9.2 Hz, 2H, ArH), 4.74 (s, 1H, CH), 4.51 (s, 1H, CH), 3.66 (s, 3H, OCH₃), 3.58 (dd, J=9.2, 1.6 Hz, 1H, CH), 3.45 (m, 2H), 2.99 (d, J=8.8 Hz, 1H), 1.99 (d, J=9.2 Hz, 1H), 1.94 (d, J=9.2 Hz, 1H); ¹³C NMR (100 MHz, DMSO-d₆) δ 153.68 (C═O), 151.38, 144.63, 141.90, 126.06, 126.03, 125.06 (q, J=270 Hz), 122.04 (q, J=31 Hz), 118.98, 115.21, 114.21, 58.01, 57.31, 56.77, 55.73, 51.11, 37.11; ¹⁹F NMR (400 MHz, DMSO-d₆) δ −60.03.

### Example 29

### Preparation of (1R,4R)—N-(tert-Butyl)-5-(4-methoxyphenyl)-2,5-diazabicyclo[2.2.1]heptane-2-carboxamide (Compound 2q)

Prepared by Method B as described in Example 6.

Yield 85.0%, White solid. Purity: (LC, tₘᵢₙ=2.86) 99.36%; UV max: 201.45, 249.45; MS (ESI) m/z 304.14 [M+H]⁺; LCMS (ESI) m/z calcd for C₁₇H₂₅N₃O₂: 304.2025 [M+H]⁺, Found: 304.2027 [M+H]⁺; ¹H NMR (400 MHz, DMSO₆) δ 6.80 (d, J=8.8 Hz, 2H, ArH), 6.53 (d, J=8.8 Hz, 2H, ArH), 5.41 (bs, 1H, NH), 4.52 (s, 1H, CH), 4.39 (s, 1H, CH), 3.65 (s, 3H, OCH₃), 3.47 (dd, J=8.8, 1.6 Hz, 1H, CH), 3.22 (s, 2H), 2.85 (d, J=8.8 Hz, 1H), 1.88 (d, J=8.8 Hz, 1H), 1.79 (d, J=8.8 Hz, 1H), 1.20 (s, 9H, (CH₃)3); ¹³C NMR (100 MHz, DMSO-d₆) δ 156.31 (C═O), 151.14, 142.06, 115.18 (2C), 114.04 (2C), 57.84, 57.35, 56.22, 55.73, 50.65, 50.29, 37.41, 29.67 (3C).

### Example 30

### Preparation of (1R,4R)-Cyclohexyl 5-(4-methoxyphenyl)-2,5-diazabicyclo[2.2.1]heptane-2-carboxylate (Compound 2r)

Prepared by Method A as described in Example 6.

Yield 96.0%, White solid. Purity: (LC, tₘᵢₙ=3.66) 98.75%; UV max: 249.45; MS (ESI) m/z 331.19 [M+H]⁺; LCMS (ESI) m/z calcd for C₁₉H₂₆N₂O₃: 331.2022 [M+H]⁺, Found: 331.2023 [M+H]⁺; ¹H NMR (400 MHz, DMSO₆) δ 6.80 (d, J=8.8 Hz, 2H, ArH), 6.56 (d, J=8.8 Hz, 2H, ArH), 4.51 (m, 1H, CH), 4.40 (s, 1H, CH), 3.65 (s, 3H, OCH₃), 3.47 (m, 1H, CH), 3.29 (m, 2H), 2.88 (m, 1H), 1.92 (m, 2H), 1.73-1.62 (m, 2H), 1.65-1.52 (m, 2H), 1.43-1.28 (m, 6H); ¹³C NMR (100 MHz, DMSO-d₆) δ 154.15 (C═O), 151.30, 141.82, 115.18 (2C), 114.12 (2C), 72.44, 58.18, 57.68, 57.29, 56.89, 56.75, 55.72, 50.96, 37.52, 31.94, 25.40, 23.51.

### Example 31

### Preparation of (1R,4R)—N-(5-Chloro-2-methoxyphenyl)-5-(3,4-dimethoxyphenyl)-2,5-diazabicyclo[2.2.1]heptane-2-carboxamide (Compound 2s)

Prepared by Method A as described in Example 6.

White-off solid. Yield 63%. Purity: (LC, tₘᵢₙ=3.43) 96.14%. UV max: 214.45, 248.45, 289.45. MS (ESI) m/z 418.19 [M+H]⁺; 416.30 [M−H]⁻. LCMS (ESI) m/z calcd for C₂₁H₂₄ClN₃O₄: 416.1377 [M−H]⁻, Found: 416.1356 [M−H]⁻. ¹H NMR (400 MHz, DMSO-d₆) δ 7.87 (bs, 1H, NH), 7.35 (m, 1H, ArH), 6.97 (m, 2H), ArH), 6.78 (d, J=9.2 Hz, 1H, ArH), 7.21 (m, 1H, ArH), 6.78 (d, J=8.8 Hz, 1H), 6.24 (d, J=2.4 Hz, 1H), 6.09 (dd, J=8.8, 2.4 Hz, 1H), 4.65 (s, 1H), 4.52 (s, 1H), 3.77 (s, 3H, OCH₃), 3.72 (s, 3H, OCH₃), 3.62 (s, 3H, OCH₃), 3.54 (d, J=7.6 Hz, 1H), 3.47 (d, J=8.8 Hz, 1H), 3.39 (d, J=9.2 Hz, 1H), 3.03 (d, J=9.2 Hz, 1H), 1.96 (d, J=9.2 Hz, 1H), 1.92 (d, J=9.2 Hz, 1H). ¹³C NMR (100 MHz, DMSO-d₆) δ 153.46 (C═O), 150.45, 148.10, 142.63, 140.83, 130.18, 124.32, 122.31, 120.05, 114.89, 112.45, 104.16, 99.25, 57.59, 57.42, 56.97, 56.80, 56.52, 55.89, 51.05, 37.37.

### Example 32

### Preparation of N-(3,4-Difluorophenyl)-3-(4-fluorophenyl)-1H-pyrrole-1-carboxamide (Compound 3a)

Prepared by Method A as described in Example 6.

Yield 82%. White-off solid. Purity: (LC, tₘᵢₙ=3.72) 98.64%. UV max: 222.45. MS (ESI) m/z 317.10 [M+H]⁺; 315.34 [M−H]⁻. LCMS (ESI) m/z calcd for C₁₇H₁₁F₃N₂O: 317.0902 [M−H]⁻. Found: 317.0883 [M+H]⁺. ¹H NMR (400 MHz, CDCl₃) δ 7.53-7.50 (m, 1H, ArH), 7.45-7.42 (m, 3H, ArH), 7.32 (bs, 1H, NH), 7.26 (m, 1H, ArH), 7.10-7.08 (m, 2H), 7.01 (t, J=8.8 Hz, 2H, ArH), 6.55 (dd, J=3.2, 1.6 Hz, 1H, ArH). ¹⁹F NMR (400 MHz, CDCl₃) δ −115.55, −134.87, −141.50.

### Example 33

### Preparation of N-(3,4-Difluorophenyl)-4-(4-methoxyphenyl)-1H-pyrazole-1-carboxamide (Compound 3b)

Prepared by Method A as described in Example 6.

Yield 67%. White solid. Purity: (LC, t min=2.67) 97.06%. UV max: 252.45. MS (ESI) m/z 319.13 [M−H]⁻. ¹H NMR (400 MHz, CDCl₃) δ 10.76 (bs, 1H, NH), 8.83 (s, 1H), 8.38 (s, 1H), 7.92-7.87 (m, 1H, ArH), 7.75 (d, J=8.4 Hz, 2H, ArH), 7.66-7.64 (m, 1H, ArH), 7.51-7.44 (m, 1H, ArH), 6.99 (d, J=8.4 Hz, 2H, ArH), 3.79 (s, 3H, OCH₃). ¹⁹F NMR (400 MHz, CDCl₃) δ −137.23, −143.97.

### Example 34

### Preparation of 1,3-bis(3,4-Difluorophenyl)urea (Compound 4a)

Prepared by Method A & B as described in Example 6.

Yield 86%. White solid. MS (ESI) m/z 285.02 [M+H]⁺; 283.10 [M−H]⁻. LCMS (ESI) m/z calcd for C₁₉H₁₉F₂N₃O₂: 360.1524 [M+H]⁺. Found: 360.1536 [M+H]⁺ and 358.1447 [M−H]⁻. ¹H NMR (400 MHz, CDCl₃) δ 8.961 (bs, 2H, NH), 7.66-7.06 (m, 2H, ArH), 7.35 (q, J=9.2 Hz, 2H, ArH), 7.14-7.11 (m, 2H, ArH). ¹³C NMR (100 MHz, CDCl₃) δ 152.85 (N—CO—N), 150.79 (dd, J=450, 12 Hz, 2C), 148.38 (dd, J=488, 13 Hz, 2C), 137.03 (q, J=2 Hz, 2C), 107.94 (d, J=17 Hz, 2C), 137.03 (q, J=3 Hz, 2C), 107.96 (d, J=21 Hz, 2C). ¹⁹F NMR (CDCl₃, 400 MHz) δ −137.44, −146.83.

### Example 35

### Preparation of 1-(3,4-Difluorophenyl)-3-(3,4,5-trifluorophenyl)urea (Compound 4b)

Prepared by Method A as described in Example 6.

Yield 82%. White solid. Purity: (LC, tₘᵢₙ=3.72) 99.35%; MS (ESI) m/z 303.09 [M+H]; 301.16 [M−H]⁻; LCMS (ESI) m/z calcd for C₁₃H₇F₆N₂O: 303.0557 [M+H]⁺, Found: 303.0566 [M+H]⁺. ¹H NMR (400 MHz, CDCl₃) δ 9.19 (bs, 1H, NH), 9.16 (bs, 1H, NH), 7.69-7.61 (m, 1H, ArH), 7.40-7.37 (m, 3H), 7.15-7.12 (m, 1H). ¹³C NMR (100 MHz, CDCl₃) δ 152.71 (C═O), 148.38, 140.67, 140.56, 130.92, 117.98, 117.79, 116.73, 111.87, 108.07, 107.85, 106.99, 106.74.

### Example 36

### Preparation of 1-(4-Chloro-3-fluorophenyl)-3-(3,4-difluorophenyl)urea (Compound 4c)

Prepared by Method A as described in Example 6.

Yield 82%. White-off solid. Purity (LC, tₘᵢₙ=3.70) 98.65%. UV 210.45, 257.45 nm. MS (ESI) m/z 301.06 [M+H]⁺; 299.18 [M−H]⁻. LCMS (ESI) m/z calcd for C₁₃H₆ClFN₂O: 301.0356. Found: 301.0373 [M+H]⁺. ¹H NMR (400 MHz, CDCl₃) δ 9.11 (bs, 1H, NH), 9.02 (bs, 1H, NH), 7.66-7.60 (m, 2H, ArH), 7.46 (t, J=8.6 Hz, 1H), 738-7.31 (m, 1H), 7.18 (dd, J=8.8, 1.6 Hz, 1H), 7.17 (m, 1H). ¹³C NMR (100 MHz, CDCl₃) δ 152.71 (C═O), 148.38, 140.67, 140.56, 130.92, 117.98, 117.79, 116.73, 111.87, 108.07, 107.85, 106.99, 106.74.

### Example 37

### Preparation of 1,3-bis(3,4,5-Trifluorophenyl)urea (Compound 4d)

Prepared by Method A as described in Example 6.

Yield 82%. White solid. Purity: (LC, tₘᵢₙ=3.66) 99.50%. UV max: 253.45, 190.45; MS (ESI) m/z 321.04 [M+H]⁺; 319.20 [M−H]⁻. LCMS (ESI) m/z calcd for C₁₃H₆F₆N₂O: 319.0306 [M−H]⁻, Found: 319.0314 [M−H]⁻. ¹H NMR (400 MHz, DMSO-d₆) δ 9.22 (bs, 2H, NH), 7.37 (dd, J=10.4, 6.4 Hz, 4H, ArH). ¹³C NMR (100 MHz, DMSO-d₆) δ 152.60 (NHC(═O)NH), 151.83 (m, 2C), 149.40 (m, 2C), 136.31 (2C), 136.16 (m, 2C), 103.38 (d, J=24 Hz, 4C). ¹⁹F NMR (DMSO-d₆, 400 MHz) δ −135.09 (4F), −169.90 (2F).

### Example 38

### Preparation of 1-(3,4-Difluorophenyl)-3-(4-methoxyphenyl)urea (Compound 4e)

Prepared by Method A as described in Example 6.

Yield 71%. Light brown solid. UV max: 190.45, 223.45. Purity (LC, tₘᵢₙ=3.08): 95.57%.

MS (ESI) m/z 279.17 [M+H]⁺; 277.25 [M−H]⁻. LCMS (ESI) m/z calcd for C₁₄H₁₂F₂N₂O₂: 277.0789 [M−H]⁻. Found: 277.0796 [M−H]⁻; 279.0957 [M+H]⁺. ¹H NMR (400 MHz, DMSO-d₆) δ 8.80 (bs, 1H, NH), 8.53 (bs, 1H, NH), 7.65 (m, 1H, ArH), 7.08 (d, J=9.0 Hz, 2H, ArH), 7.07 (m, 1H, ArH), 7.09 (m, 1H, ArH), 6.85 (d, J=9.0 Hz, 2H, ArH), 3.70 (s, 3H, OCH3). ¹⁹F NMR (DMSO-d₆, 400 MHz) δ −137.57, −147.50.

### Example 39

### Preparation of 1,3-bis(4-Methoxyphenyl)urea (Compound 4f)

Prepared by Method A as described in Example 6.

Yield 81%. White-off solid. Purity: (LC, tₘᵢₙ=2.80) 97.78%. UV max: 190.45, 223.45. MS (ESI) m/z 273.17 [M+H]⁺; 295.14 [M+Na]⁺. LCMS (ESI) m/z calcd for C₁₅H₁₆N₂O₃: 271.1083 [M−H]⁻. Found: 271.1084 [M−H]⁻; 273.1024 [M+H]⁺. ¹H NMR (400 MHz, CDCl₃) δ 9.34 (bs, 2H, NH), 7.22 (d, J=8.4 Hz, 4H, ArH), 6.88 (d, J=8.4 Hz, 4H, ArH), 3.80 (s, 6H, 2 (OCH₃)).

### Example 40

### Preparation of N-(3,4-Difluorophenyl)-4-fluoro-1H-indole-1-carboxamide (Compound 4g)

Prepared by Method A as described in Example 6.

Yield 77%. Yellowish solid. Purity: (LC, tₘᵢₙ=3.66) 97.56%. UV max: 190.45, 223.45.

MS (ESI) m/z 291.13 [M+H]⁺; 289.15 [M−H]⁻. LCMS (ESI) m/z calcd for C₁₅H₉F₃N₂O: 289.0589 [M−H]⁻. Found: 289.0588 [M−H]⁻; 291.0719 [M+H]⁺. ¹H NMR (400 MHz, CDCl₃) δ 7.82 (d, J=8.4 Hz, 1H, ArH), 7.54 (dd, J=6.8, 1.6 Hz, 1H, ArH), 7.44 (d, J=4.0 Hz, 1H, ArH), 7.43 (bs, 1H, NH), 7.23 (m, 1H, ArH), 7.10 (m, 2H, ArH), 6.89 (t, J=8.8 Hz, 1H, ArH), 6.73 (d, J=3.8 Hz, 1H, ArH). ¹⁹F NMR (CDCl₃, 400 MHz) δ −121.08, −134.93, −141.53.

### Example 41

### Preparation of 4-Cyano-N-(3,4-difluorophenyl)-1H-indole-1-carboxamide (Compound 4h)

Prepared by Method A as described in Example 6.

Yield 81%. White solid. Purity (LC, tₘᵢₙ=3.58) 96.39%. MS (ESI) m/z 298.13 [M+H]⁺; 296.38 [M−H]⁻. LCMS (ESI) m/z calcd for C₁₆H₉F₂N₃O: 296.0635 [M−H]⁻. Found: 296.0674 [M−H]⁻. ¹H NMR (400 MHz, DMSO-d₆) δ 10.49 (bs, 1H, NH), 8.54 (d, J=8.4 Hz, ArH), 8.29 (d, J=3.6 Hz, ArH), 7.81 (m, 1H, ArH), 7.77 (d, J=8.4 Hz, 1H, ArH), 7.51 (d, J=8.4 Hz, 1H, ArH), 7.47 (m, 2H, ArH), 6.96 (d, J=3.6 Hz, ArH). ¹³C NMR (100 MHz, DMSO-d₆) δ 149.66 (N—CO—N), 149.47 (dd, J_{F-F}=243, 13 Hz), 146.45 (dd, J_{F-F}=241, 12 Hz), 135.53, 131.38, 129.30, 127.84, 124.64, 120.57, 128.24, 128.09 (d, J_{F-F}=8 Hz), 127.75 (q, J_{F-F}=3 Hz), 110.52, 110.31, 104.61, 102.86. ¹⁹F NMR (CDCl₃, 400 MHz) δ −137.12, −143.87.

### Example 42

### Preparation of 3,4-Difluorophenyl (3,4-difluorophenyl)carbamate (Compound 4i)

Under argon atmosphere, to a solution of triphosgene (1.48 g, 5 mmol) in 10 mL of anhydrous THF, 3,4-difluorophenol (650 mg, 5 mmol) in 3 mL of THF was added slowly at 0° C. to the solution and triethylamine (0.7 mL, 5 mmol) was droppwisly added in the solution. The resulting mixture was stirred at the same temperature for 10 min and allowed to heat to reflux for 30 min. After cooling the flask, 3,4-difluoroaniline (645 mg, 5 mmol) was added to the solution and stirred overnight at room temperature. The reaction mixture was concentrated under reduced pressure and then dissolved into 50 mL of ethyl acetate, washed with water (50 mL), saturated NaHCO₃ (20 mL), water (30 mL), 3 N HCl (20 mL) and water (30 mL). The organic layer was dried over anhydrous MgSO₄, and concentrated under reduced pressure, purified with flash column chromatography using EtOAc/Hexane (1/3, v/v) as an eluent to produce desired product. Yield 72.9% as a white solid. Purity: 98.37%; UV max: 190.45, 230.45; MS (ESI) m/z 285.99 [M+H]+, 283.95 [M−H]⁻; LCMS (ESI) m/z calcd for C₁₃H₇F₄NO₂: 286.0491 [M+H]⁺, Found: 286.0479 [M+H]⁺. ¹H NMR (400 MHz, CDCl₃) δ 7.48 (t, J=8.8 Hz, 1H, NH), 7.18 (q, J=8.8 Hz, 1H, ArH), 7.10-7.08 (m, 1H, ArH), 7.08-7.06 (m, 1H, ArH), 7.06-7.02 (m, 1H, ArH), 6.97-6.95 (s, 2H, ArH). ¹⁹F NMR (400 MHz, CDCl₃) δ −134.33, −135.04, −140.65, −142.79.

### Example 43

### Preparation of N,2-bis(3,4-Difluorophenyl)acetamide (Compound 4j)

Purity (LC, t_{R} 3.28 min): 96.97%; UV max: 221.45; MS (ESI) m/z 283.98 [M+H]⁺; 282.06 [M−H]⁻; LCMS (ESI) m/z calcd for C₁₄H₉F₄NO: 284.0699 [M+H]⁺, Found: 284.0691 [M+H]⁺. ¹H NMR (400 MHz, DMSO-d₆) δ 10.43 (bs, 1H), 8.11 (dq, J=7.6, 2.4 Hz, 1H), 7.43-7.36 (m, 3H), 7.30-7.27 (m, 1H), 7.17-7.14 (m, 1H), 3.68 (s, CH₃, 2H). ¹³C NMR (100 MHz, DMSO-d₆) δ 169.23 (C═O), 150.78 (dd, J_{C-F}=244.0, 12.5 Hz), 150.60 (dd, J_{C-F}=46.0, 12.5 Hz), 148.19 (dd, J_{C-F}=46.0, 12.5 Hz), 146.92 (dd, J_{C-F}=240.0, 12.5 Hz), 136.59 (q, J_{C-F}=6.0, 3.0 Hz), 133.66 (q, J_{C-F}=6.0, 3.0 Hz), 126.64 (q, J_{C-F}=6.0, 3.0 Hz), 118.84 (d, J_{C-F}=17.0 Hz), 118.84 (d, J_{C-F}=17.0 Hz), 118.04 (d, J_{C-F}=17.0 Hz), 117.72 (d, J_{C-F}=17.0 Hz), 115.87 (q, J_{C-F}=3.0 Hz), 108.65 (d, J_{C-F}=21.0 Hz), 42.27. ¹⁹F NMR (400 MHz, DMSO-d₆) δ −137.14, −139.25, −141.79, −144.74.

### Example 44

### Preparation of S-(3,4-difluorophenyl) (3,4-difluorophenyl)carbamothioate (Compound 4k)

Under argon atmosphere, to a solution of triphosgene (594 mg, 2 mmol) in 10 mL of anhydrous THF, 3,4-difluorothiophenol (292 mg, 2 mmol) in 3 mL of THF was added slowly at 0° C. to the solution and triethylamine (0.42 mL, 3 mmol) was drop wisely added in the solution. The resulting mixture was stirred at the same temperature for 10 min and allowed to heat to reflux for 30 min. After cooling the flask, the solution of 3,4-difluoroaniline (258 mg, 2 mmol) in 2 mL anhydrous THF was added to the mixture and stirred overnight at room temperature. The reaction mixture was concentrated under reduced pressure and then dissolved into 30 mL of ethyl acetate, washed with water (30 mL), brine (30 mL). The organic layer was dried over anhydrous MgSO₄, and concentrated under reduced pressure, purified with flash column chromatography using EtOAc/Hexane (1/3, v/v) as an eluent to produce desired product.

Yield 89%. White solid. Purity: 97.90%; UV max: 195.45, 248.45 nm; MS (ESI) m/z 302.02 [M+H]⁺; LCMS (ESI) m/z calcd for C₁₃H₇F₄NOS: 302.0263 [M+H]⁺, Found: 302.0260 [M+H]⁺. ¹H NMR (400 MHz, CDCl₃) δ 7.49-7.40 (m, 1H, ArH), 7.33-7.30 (m, 1H, ArH), 7.27-7.21 (m, 1H, ArH), 7.18 (bs, 1H, NH), 7.12 (q, J=9.2 Hz, 1H, ArH), 7.01-6.99 (m, 1H, ArH); ¹⁹F NMR (400 MHz, CDCl₃) δ −134.33, −135.04, −140.65, −142.79.

### Example 45

### Preparation of bis((1R,4R)-5-(4-Methoxyphenyl)-2,5-diazabicyclo[2.2.1]heptan-2-yl)methanone (Compound 5)

A 100 mL, oven-dried, two-necked, round-bottomed flask is charged with a Teflon-coated magnetic oval stir bar and coupled with a 50 mL dropping funnel. Both the dropping funnel and the round-bottomed flask are sealed with a rubber septum. Under nitrogen atmosphere, DJ-VIII-038R (312 mg, 1.52 mmol) in 3 mL of THF was added slowly at 0° C. to a solution of triphosgene (226 mg, 0.76 mmol) in 10 mL of dry THF. The resulting mixture was stirred at the same temperature for 10 min and allowed to stir at room temperature for another 30 min. After completion of reaction, the reaction mixture was added to crushed ice and extracted with ethyl acetate. The organic layer was dried over anhydrous MgSO₄ and concentrated under reduced pressure to purify by silica gel chromatography (EtOAc/n-hexane=1:1) or (hexane/Acetone=3:1, v/v) to afford the target compound as yellowish solid (5, DJ-VIII-80R, Yield 73%) and compound 5a (1R,4R)-5-(4-methoxyphenyl)-2,5-diazabicyclo[2.2.1]heptane-2-carboxylic anhydride, Yield 7% as a brown solid.

For compound 5, Yield 83%. Yellowish solid. Purity: (LC, tₘᵢₙ=3.13) 97.64%. MS (ESI) m/z 435.31 [M+H]⁺. LCMS (ESI) m/z calcd for C₂₅H₃₀N₄O₃: 435.2396 [M+H]⁺. Found: 435.2378 [M+H]⁺. ¹H NMR (400 MHz, DMSO-d6) δ 6.78 (d, J=8.6 Hz, 4H, ArH), 6.50 (d, J=8.6 Hz, 4H, ArH), 4.35 (s, 2H), 4.19 (s, 2H), 3.65 (s, 6H, (OCH₃)2), 3.45 (d, J=8.2 Hz, 2H), 3.32 (d, J=8.2 Hz, 2H), 3.01 (d, J=8.8 Hz, 2H), 3.00 (d, J=8.8 Hz, 2H), 1.80 (bs, 4H). ¹³C NMR (100 MHz, DMSO-d6) δ 159.85 (N—CO—N), 151.13 (2C), 141.87 (2C), 115.21 (4C), 113.99 (4C), 58.02 (2C), 57.36 (2C), 56.57 (2C), 55.72 (2C, (OCH₃)₂), 52.69 (2C), 36.39 (2C).

### Example 46

### TMZ-Induced Cell Death with Series 2 Compounds

The activity of series 2 compounds with PFI-3 to sensitize GBM cells to TMZ-induced cell death was assessed with TMZ (FIG. 2A) and without TMZ (FIG. 2B). LN229 GBM cells were treated with PFI-3 or the indicated series 2 compounds (2 μM) in combination with TMZ (50 μm). Control cells were treated with vehicle (DMSO). Treatment with TEDs alone had no effect on cell viability. Cell death was determined by ELISA at 72 hr which quantifies histone-complexed DNA fragments. Results were normalized to the cell death-inducing activity of PFI-3 in combination with TMZ.

### Example 47

### TMZ-Induced Cell Death with Series 3, 4 and 5 Compounds

The activity of series 3, 4 and 5 compounds with PFI-3 to sensitize GBM cells to TMZ-induced cell death was assessed with TMZ (FIG. 3A) and without TMZ (FIG. 3B). LN229 GBM cells were treated with PFI-3 or the indicated series 3, 4, or 5 compounds (2 M) in combination with TMZ (50 μM). Control cells were treated with vehicle (DMSO). Treatment with TEDs alone had no effect on cell viability. Cell death was determined by ELISA at 72 hr which quantifies histone-complexed DNA fragments. Results were normalized to the cell death-inducing activity of PFI-3 in combination with TMZ.

### Example 48

### 3-Dimensional Cell Culture Assay

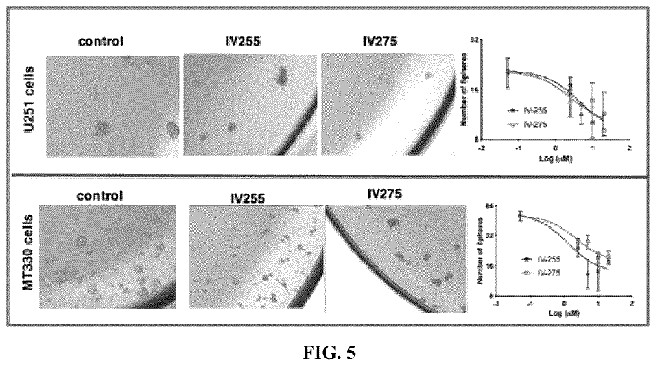

GBM cells were plated in nonadherent 96 well plates (1×104 cells/well) and grown in supplemented neural basal media. At one day of plating cells were treated with two of the lead compounds (IV-255 and IV-257), and the number of tumor spheres (colonies) were enumerated after 7 days of culture. As shown in FIG. 5, IV-255 and IV-257 reduced the number of tumor spheres by a log-fold difference.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

The foregoing written specification and following examples are considered to be sufficient to enable one skilled in the art to practice the invention. Various modifications of the invention in addition to those shown and described herein will become apparent to those skilled in the art from the foregoing description and following examples and fall within the scope of the appended claims. The disclosures of all citations in the specification are expressly incorporated herein by reference.

To comply with written description and enablement requirements, all references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material. Incorporated by reference are the following references:

The various features and processes described above may be used independently of one another or may be combined in various ways. All possible combinations and subcombinations are intended to fall within the scope of this disclosure. In addition, certain method or process blocks may be omitted in some implementations. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in serial, in parallel, or in some other manner. Blocks or states may be added to or removed from the disclosed example embodiments. The example systems and components described herein may be configured differently than described. For example, elements may be added to, removed from, or rearranged compared to the disclosed example embodiments.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements, and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

While certain example embodiments have been described, these embodiments have been presented by way of example only, and are not intended to limit the scope of the inventions disclosed herein. Thus, nothing in the foregoing description is intended to imply that any particular feature, characteristic, step, module, or block is necessary or indispensable. Indeed, the novel methods and systems described herein may be embodied in a variety of other forms; furthermore, various omissions, substitutions and changes in the form of the methods and systems described herein may be made without departing from the spirit of the inventions disclosed herein. The accompanying claims and their equivalents are intended to cover such forms or modifications as would fall within the scope and spirit of certain of the inventions disclosed herein.

All references cited herein are incorporated herein by reference in their entirety. To the extent publications and patents or patent applications incorporated by reference contradict the disclosure contained in the specification, the specification is intended to supersede and/or take precedence over any such contradictory material.

What is claimed is:

1. A compound selected from the group consisting of

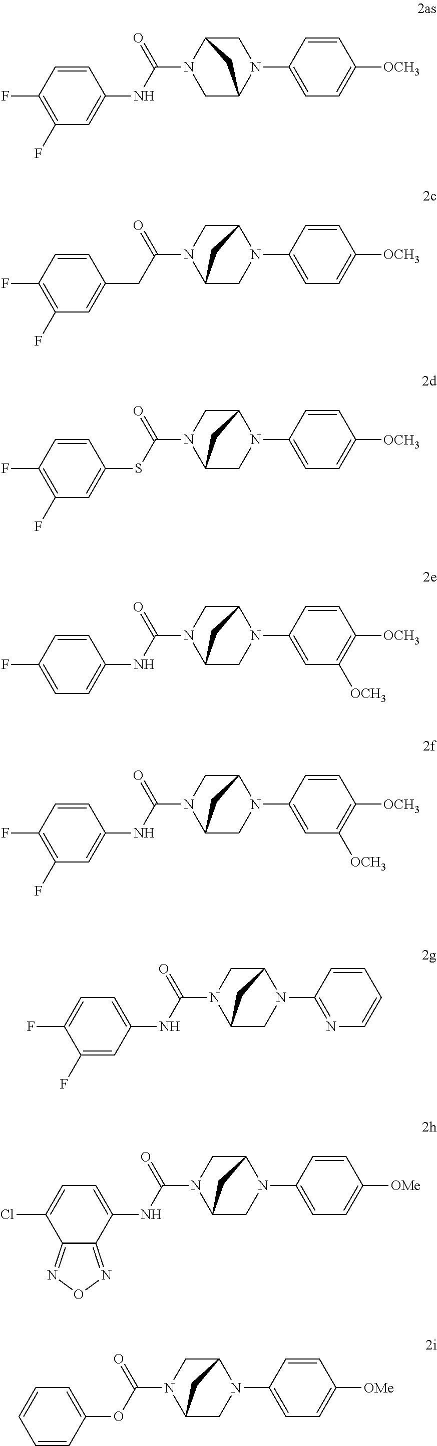

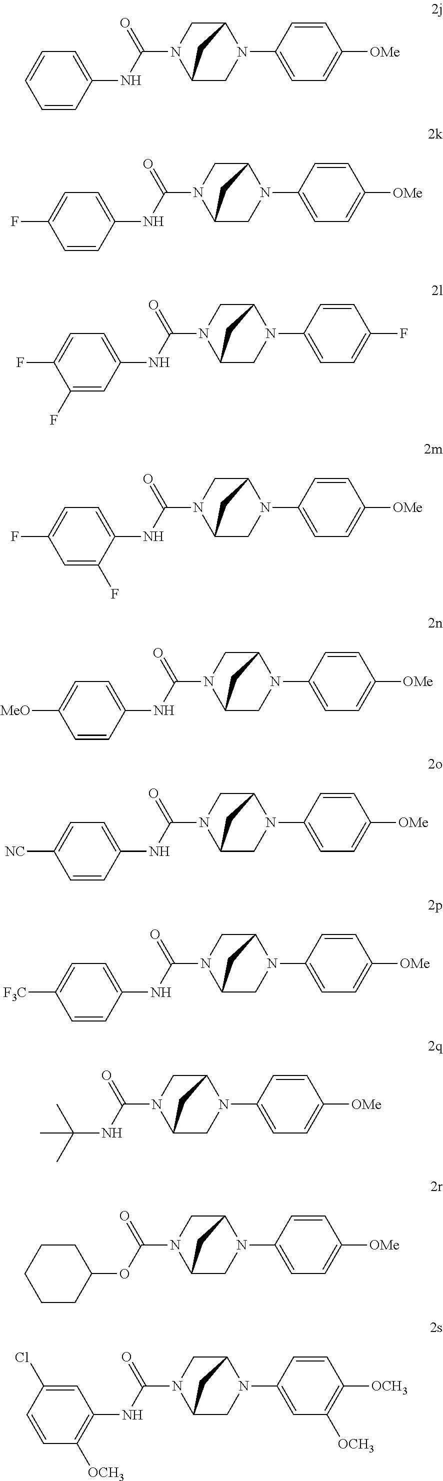

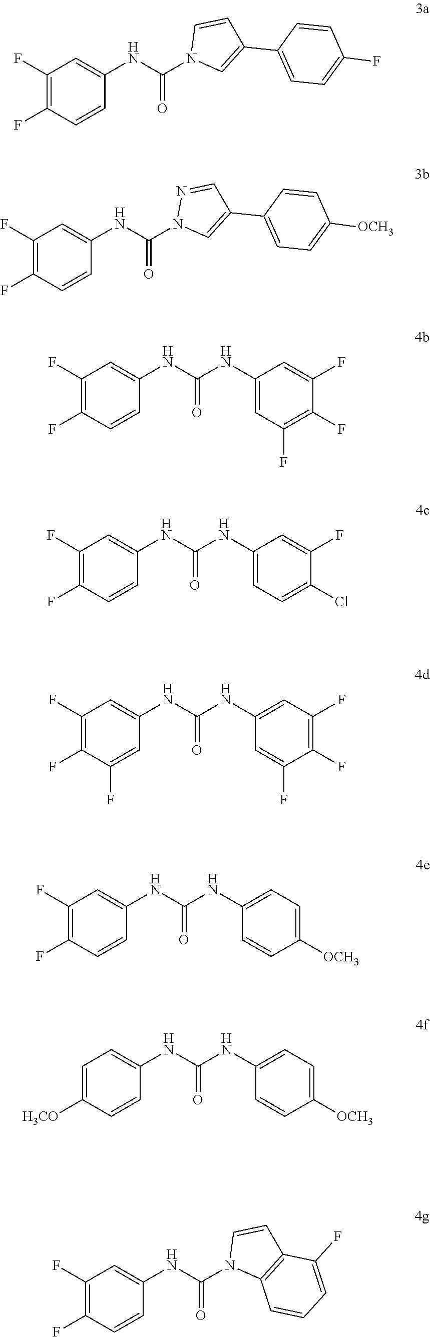

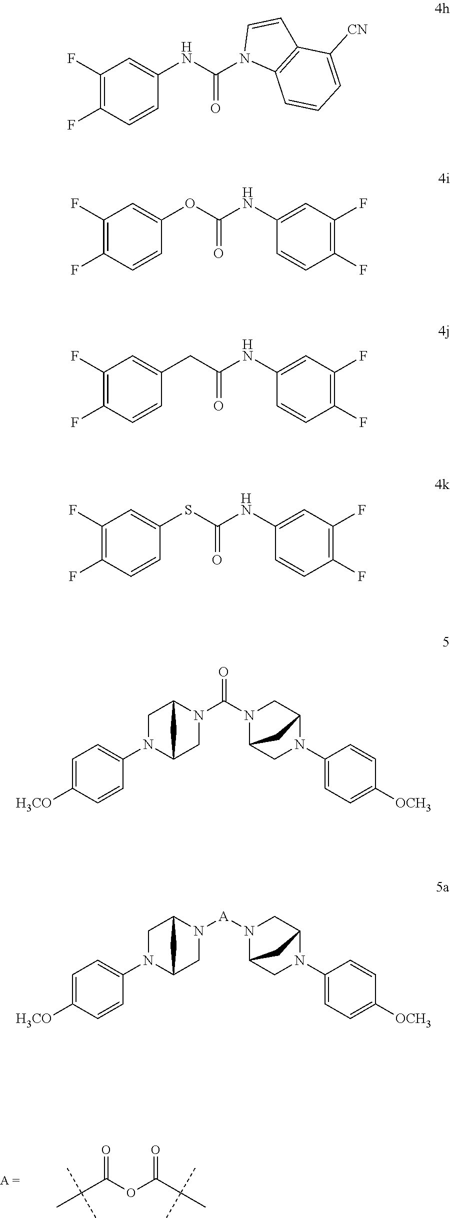

2. A pharmaceutical composition comprising a compound selected from the group consisting of: 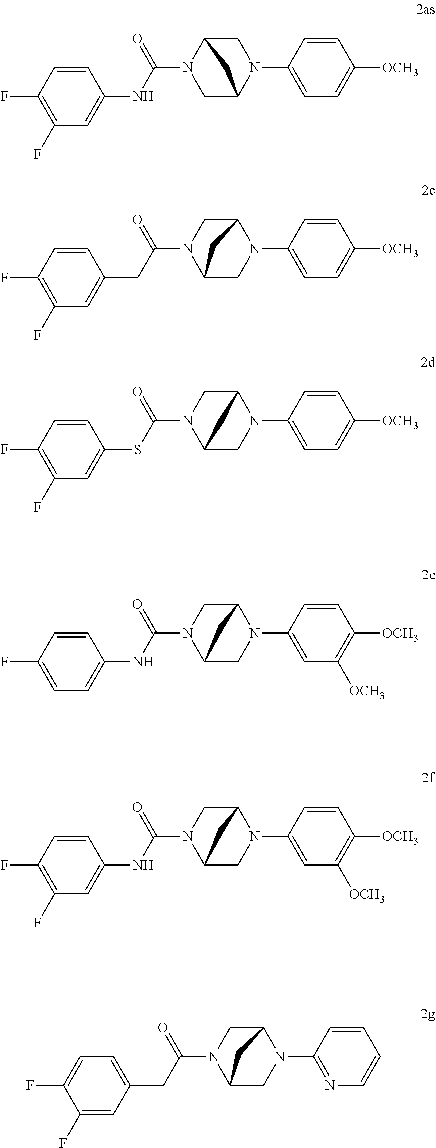 [SMILES: COc1ccc(N2C[C@@H]3C[C@H]2CN3C(=O)Nc2ccc(F)c(F)c2)cc1 ; COc1ccc(N2C[C@H]3C[C@@H]2CN3C(=O)Cc2ccc(F)c(F)c2)cc1 ; COc1ccc(N2C[C@H]3C[C@@H]2CN3C(=O)Sc2ccc(F)c(F)c2)cc1 ; COc1ccc(N2C[C@H]3C[C@@H]2CN3C(=O)Nc2ccc(F)cc2)cc1OC ; COc1ccc(N2C[C@H]3C[C@@H]2CN3C(=O)Nc2ccc(F)c(F)c2)cc1OC ; O=C(Cc1ccc(F)c(F)c1)N1C[C@H]2C[C@@H]1CN2c1ccccn1] 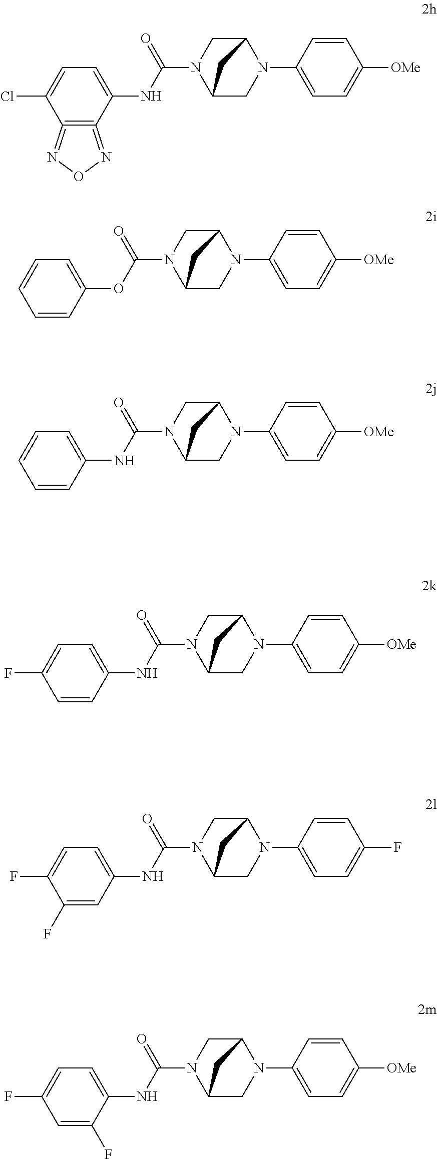 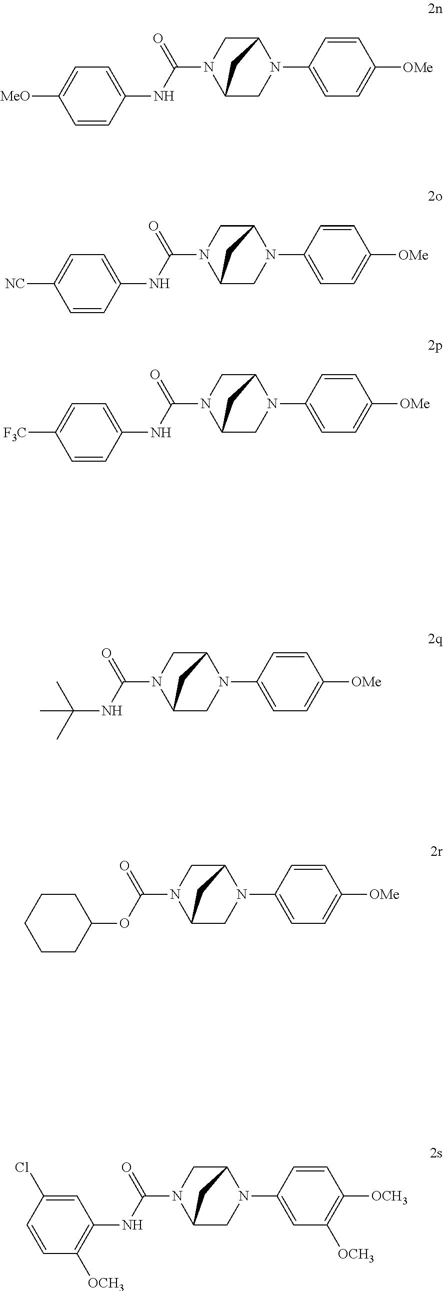 [SMILES: COc1ccc(NC(=O)N2C[C@H]3C[C@@H]2CN3c2ccc(OC)cc2)cc1 ; COc1ccc(N2C[C@H]3C[C@@H]2CN3C(=O)Nc2ccc(C#N)cc2)cc1 ; COc1ccc(N2C[C@H]3C[C@@H]2CN3C(=O)Nc2ccc(C(F)(F)F)cc2)cc1 ; COc1ccc(N2C[C@H]3C[C@@H]2CN3C(=O)NC(C)(C)C)cc1 ; COc1ccc(N2C[C@H]3C[C@@H]2CN3C(=O)OC2CCCCC2)cc1 ; COc1ccc(Cl)cc1NC(=O)N1C[C@H]2C[C@@H]1CN2c1ccc(OC)c(OC)c1] 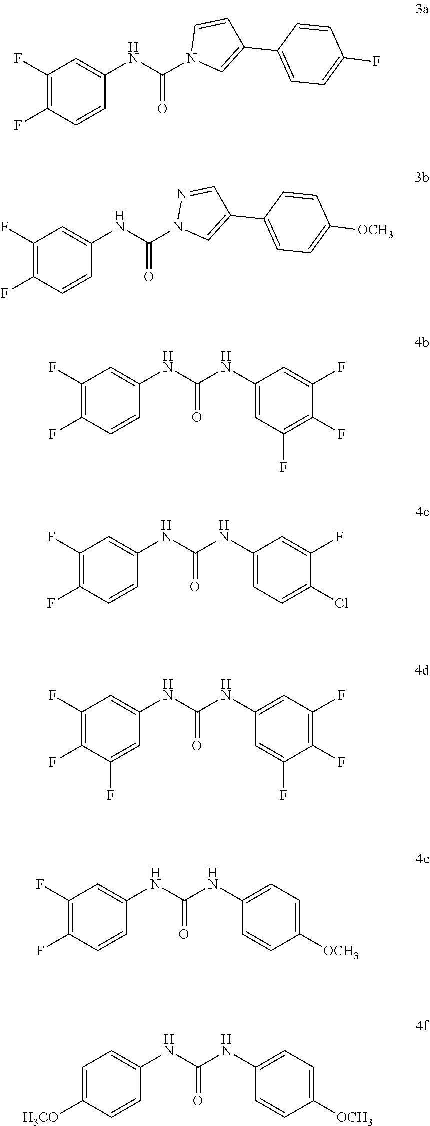 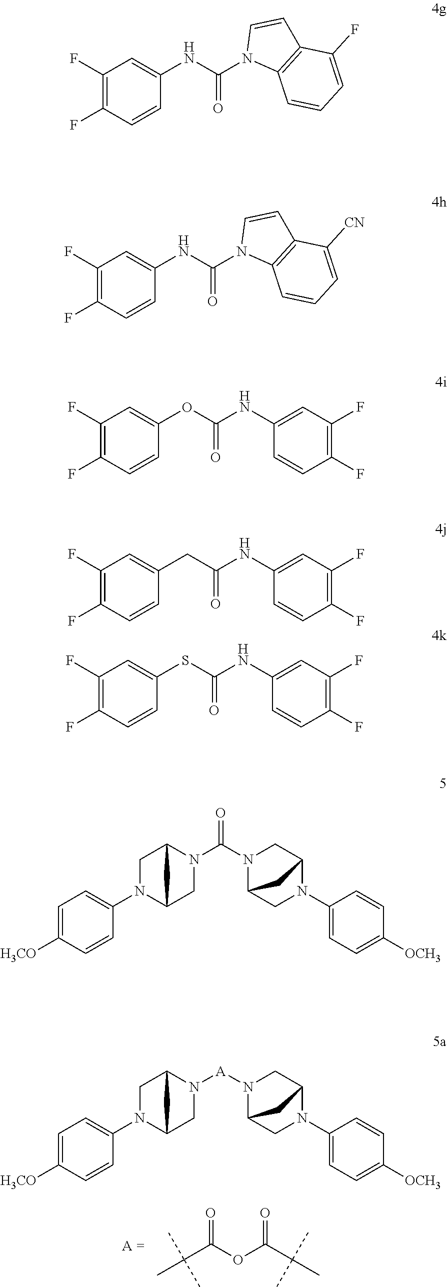
a prodrug thereof, or a pharmaceutically acceptable salt thereof.

3. The pharmaceutical composition of claim 2, further comprising at least one pharmaceutically acceptable carrier.

4. The pharmaceutical composition of claim 3, wherein the pharmaceutical composition further comprises a DNA alkylating agent.

5. The pharmaceutical composition of claim 4, wherein the DNA alkylating agent is temozolomide.

6. The pharmaceutical composition of claim 2, wherein the compound is selected from the group consisting of: 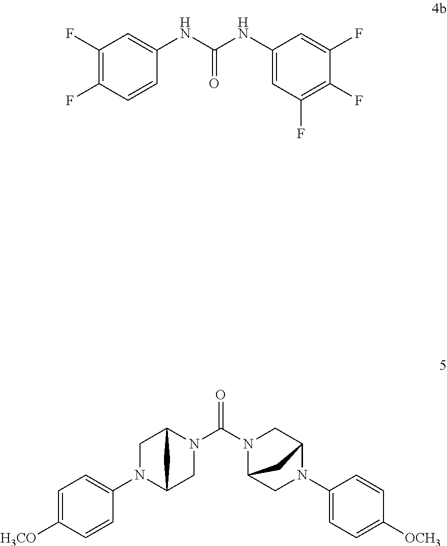 [SMILES: O=C(Nc1ccc(F)c(F)c1)Nc1cc(F)c(F)c(F)c1 ; COc1ccc(N2C[C@H]3C[C@@H]2CN3C(=O)N2C[C@H]3C[C@@H]2CN3c2ccc(OC)cc2)cc1]
a prodrug thereof, or a pharmaceutically acceptable salt thereof.

7. The pharmaceutical composition of claim 2, wherein the compound is: 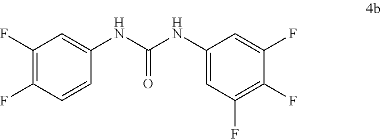 [SMILES: O=C(Nc1ccc(F)c(F)c1)Nc1cc(F)c(F)c(F)c1]
a prodrug thereof, or a pharmaceutically acceptable salt thereof.

8. The pharmaceutical composition of claim 2, wherein the compound is: 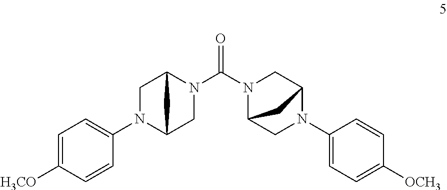 [SMILES: COc1ccc(N2C[C@H]3C[C@@H]2CN3C(=O)N2C[C@H]3C[C@@H]2CN3c2ccc(OC)cc2)cc1]
a prodrug thereof, or a pharmaceutically acceptable salt thereof.

9. A method of treating cancer in a subject in need thereof by administering a pharmaceutical composition comprising a therapeutically effective amount of at least one of the following compounds: 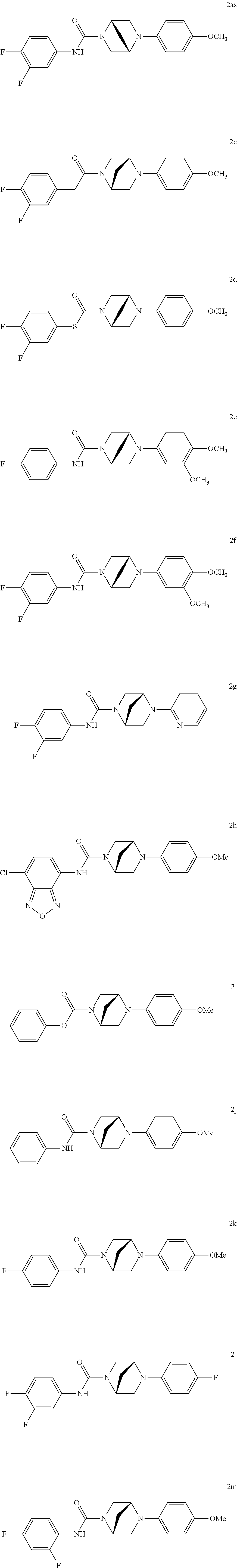 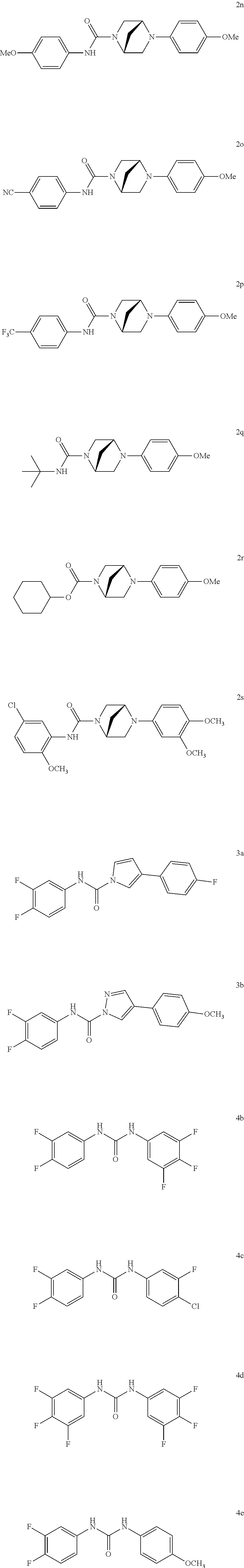 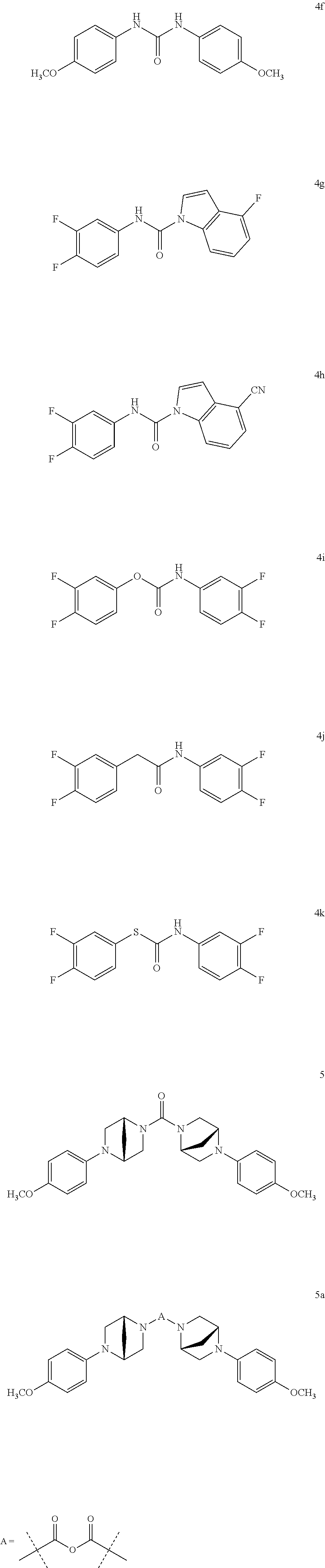
a prodrug thereof, or a pharmaceutically acceptable salt thereof.

10. The method of claim 9, wherein the cancer is glioblastoma.

11. The method of claim 10, wherein the glioblastoma is temozolomide-resistant glioblastoma.

12. The method of claim 9 wherein the pharmaceutical composition further comprises a therapeutically effective amount of a DNA-alkylating agent.

13. The method of claim 12 wherein the DNA-alkylating agent is temozolomide.

14. The method of claim 13 wherein the cancer is glioblastoma.

15. The method of claim 14 wherein the glioblastoma is temozolomide-resistant glioblastoma.
